(19) United States
(12) Patent Application Publication
(10) Pub. No.: US 2024/0261224 A1
(43) Pub. Date: Aug. 8, 2024

(54) FORMULATION OF A PAN-JAK INHIBITOR

(71) Applicant: KINASET THERAPEUTICS, INC., MEDFIELD, MA (US)

(72) Inventors: Frazer Giles Morgan, Chippenham (GB); Rebecca Annabel Louise Greenan, Chippenham (GB); Martin James Oliver, Chippenham (GB); Christopher D. O’Brien, Medfield, MA (US)

(21) Appl. No.: 18/526,352

(22) Filed: Dec. 1, 2023

Related U.S. Application Data
(60) Provisional application No. 63/597,934, filed on Nov. 10, 2023.
(60) Provisional application No. 63/501,086, filed on May 9, 2023.
(60) Provisional application No. 63/385,847, filed on Dec. 2, 2022.

(51) Int. Cl. A61K 9/14; A61K 9/00; A61K 9/48; A61K 31/4545; A61P 11/06

(52) U.S. Cl. CPC A61K 9/145; A61K 9/0075; A61K 9/143; A61K 9/485; A61K 9/4858; A61K 31/4545; A61P 11/06

(57) ABSTRACT

The present disclosure relates to formulations comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and methods of preparation.

### RELATED APPLICATIONS

This application claims priority to U.S. Provisional Application No. 63/597,934 filed on Nov. 10, 2023, U.S. Provisional Application No. 63/501,086 filed on May 9, 2023, and U.S. Provisional Application No. 63/385,847 filed on Dec. 2, 2022, each of which is hereby incorporated herein by reference in its entirety.

### BACKGROUND

WO 2011/051452 discloses compounds that are useful as Janus kinase inhibitors, which include JAK1, JAK2, JAK3, and TYK2 inhibitors. The compounds disclosed therein have utility in the treatment of various diseases, including respiratory indications such as asthma and COPD, as well as other inflammatory processes that may be associated with eosinophilic or non-eosinophilic inflammation.

Drugs for the treatment of respiratory diseases are frequently administered via dry powder inhalation devices. Formulating respiratory drugs as dry powders with inhalation excipients such as lactose is complicated and unpredictable. There is a continuing need for stable dry powder formulations that exhibit desirable bioavailability and physical properties. Physical characteristics are important for efficient handling and processing of the drug substance, to ensure that an effective dose is delivered to the correct part of the lung, and that the drug is effective in treating respiratory diseases. Different formulation techniques are known in the art and can be applied to drug compounds to produce inhalation powders having the desired drug delivery properties.

Disclosed herein are formulation methods and formulations of (S)-3-(3-(1-methyl-2-oxo-5-(pyrazolo[1,5-a]pyridine-3-yl)-1H-imidazo[4,5-b]pyridine-3(2H)-yl)piperidin-1-yl)-3-oxopropanenitrile, having the structure shown below and known herein as Compound I.

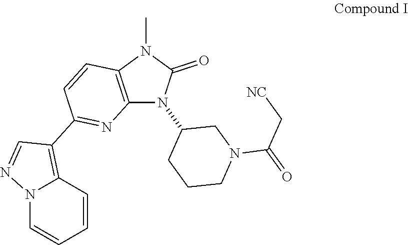

Compound I is also known as (S)-3-(3-(1-methyl-2-oxo-5-(pyrazolo[1,5-a]pyridine-3-yl)-1,2-dihydro-3H-imidazo[4,5-b]pyridine-3-yl)piperidin-1-yl)-3-oxopropanenitrile.

As discussed above, the ongoing need to treat asthma and COPD, as well as inflammatory processes associated with eosinophilic inflammation or non-eosinophilic inflammation, coupled with the potent JAK inhibitor activity of Compound I, demonstrates that there is a need for chemically stable formulations of Compound I that are suitable for use on a commercial scale. The formulations of Compound I disclosed herein meet this and other needs.

### SUMMARY

The present disclosure relates to new formulations and the methods used to make them. Provided herein are methods and formulations comprising Compound I:

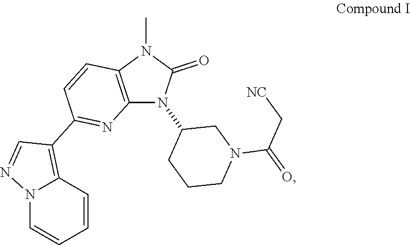

or a pharmaceutically acceptable salt, solvate, clathrate, or co-crystal thereof, as well as lactose and magnesium stearate.

In particular, the present disclosure provides, inter alia, methods and formulations comprising Compound I:

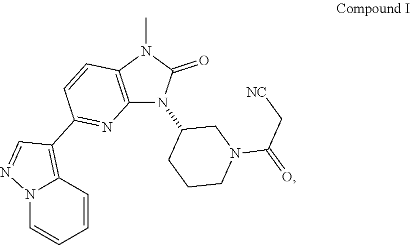

or a pharmaceutically acceptable salt thereof, lactose, and magnesium stearate. The formulations are useful in the treatment of asthma and COPD.

### BRIEF DESCRIPTION OF THE DRAWINGS

FIG. 1 is the sampling plan used for the determination of drug content and content uniformity.

FIG. 2 shows the delivery efficiency data (delivered dose) for 2 mg formulations comprising Compound I.

FIG. 3 shows the delivery efficiency data (delivered dose) for 4 mg formulations comprising Compound I.

FIG. 4 shows the phase 1 clinical trial design for formulations comprising Compound I.

FIG. 5 shows the study of the change in FeNO from Day 1 to Day 17 for patients administered formulations comprising Compound I (0.6 mg QD, 2.0 mg QD, 4.0 mg BID, and 8.0 mg BID). It is noted that the patients are characterized with mild asthma and are naïve to inhaled corticosteroid (ICS) therapy.

FIG. 6 shows the proportion of patients achieving pre-defined threshold FeNO reductions from Day 1 pre-dose to anytime on the final day of dosing (Day 10). It is noted that the patients are characterized with mild asthma and are naïve to inhaled corticosteroid (ICS) therapy.

FIG. 7a and FIG. 7b show the study of the change in FeNO from Day 1 to Day 17 for patients administered formulations comprising Compound I (4.0 mg BID). It is noted that the patients are characterized with moderate to severe asthma and use background ICS/LABA therapy.

FIG. 8 shows a comparison of FeNO reductions in Th2-high patients with moderate to severe asthma administered formulations comprising Compound I (4.0 mg BID) or formulations comprising TD-8236 (1.5 mg QD).

FIG. 9 shows the mean placebo-corrected change in FeNO from Day 1 pre-dose to the final day of dosing (Day 10) for ITT and PP patient populations administered formulations comprising Compound I (4.0 mg BID). It is noted that the patients are characterized with moderate to severe asthma and use background ICS/LABA therapy.

FIG. 10 shows the mean placebo-corrected change in FeNO from Day 1 pre-dose to the final day of dosing (Day 10): blood eosinophil subgroup populations. It is noted that the patients are characterized with moderate to severe asthma and use background ICS/LABA therapy.

FIG. 11 shows the proportion of subjects achieving pre-defined threshold FeNO reductions from Day 1 pre-dose to anytime on the final day of dosing (Day 10). It is noted that the patients are characterized with moderate to severe asthma and use background ICS/LABA therapy.

### DETAILED DESCRIPTION

Disclosed herein are formulations comprising (S)-3-(3-(1-methyl-2-oxo-5-(pyrazolo[1,5-a]pyridine-3-yl)-1H-imidazo[4,5-b]pyridine-3(2H)-yl)piperidin-1-yl)-3-oxopropanenitrile (Compound I):

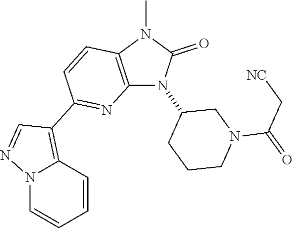

and methods for making same. In some embodiments, the formulation comprises: about 0.5-11 wt % of Compound I; about 87.0-99.0 wt % lactose; and about 0.5-2.0 wt % magnesium stearate.

The formulations and methods of preparation disclosed herein are unexpected in that they result in blends of Compound I, magnesium stearate, and lactose that have sufficient drug content and homogeneity (content uniformity). This discovery led to clinical investigations of formulations comprising Compound I, lactose, and magnesium stearate. Prior to the instant discovery, the lack of homogeneity and low drug content in existing formulations of Compound I precluded clinical studies of Compound I.

Also provided herein are methods of treating respiratory conditions (e.g., COPD and asthma) with formulations comprising Compound (I), lactose, and magnesium stearate. These treatment methods are unexpected in that they demonstrate a clinically meaningful effect on eosinophilic inflammation in asthma patients.

An additional unexpected discovery is that formulations comprising Compound (I), lactose, and magnesium stearate provide clinically relevant FeNO reduction at low doses delivered by a single capsule. In contrast, other JAK inhibitors require higher API dosing achieved by administering a high number of capsules. Thus, also provided herein are methods of reducing fractional exhaled nitric oxide (FeNO) with formulations comprising Compound (I), lactose, and magnesium stearate.

### Definitions

It is to be understood that the terminology used herein is for the purpose of describing particular embodiments only and is not intended to be limiting.

Unless defined otherwise, all technical and scientific terms used herein have the same meaning as commonly understood by one of ordinary skill in the art to which the disclosure pertains. Although other methods and materials similar, or equivalent, to those described herein can be used in the practice of the present disclosure, the preferred materials and methods are described herein.

As used in this specification and the appended claims, the singular forms “a,” “an” and “the” include plural referents unless the context clearly dictates otherwise. Thus, for example, reference to “an excipient” includes a combination of two or more such excipients, reference to “a glidant” includes one or more glidants, or mixtures of glidants, reference to “a filler” includes one or more fillers, or mixtures of fillers, and the like. Unless specifically stated or obvious from context, as used herein, the term “or” is understood to be inclusive and covers both “or” and “and.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless specifically stated or obvious from context, as used herein, the term “substantially” is understood as within a narrow range of variation or otherwise normal tolerance in the art. Substantially can be understood as within 5%, 4%, 3%, 2%, 1%, 0.5%, 0.1%, 0.05%, 0.01% or 0.001% of the stated value.

“Subject” or “patient” refers to any animal, such as a mammal. Examples of mammals include, but are not limited to, cows, horses, sheep, pigs, cats, dogs, mice, rats, rabbits, guinea pigs, non-human primates (NHPs) such as monkeys or apes, humans, etc., including a human.

“Therapeutically-effective amount” or “effective amount” refers to an amount that is effective to elicit the desired biological or medical response, including the amount of a compound that, when administered to a subject for treating a disease, is sufficient to affect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (such as additive or synergistic effects) of the compounds.

As used herein, the terms “treat,” “treatment,” and “treating” are defined as the application or administration of a therapeutic agent or a formulation, to a subject, or application or administration of a therapeutic agent or formulation to an isolated tissue or cell line from a subject (e.g., for diagnosis or ex vivo applications), who has a respiratory disorder. Such treatments may be specifically tailored or modified, based on knowledge obtained from the field of pharmacogenomics.

As used herein, the terms “formulation,” “pharmaceutical formulation,” and “dry powder pharmaceutical formulation” refer to a blend, aggregation, solution or other combination of materials which includes an active pharmaceutical ingredient (API). In a non-limiting example, the formulation includes an API and one or more excipients.

As used herein, the term “combining” or “combined” is used to refer to the action of adding two or more components together to form a mixture or preblend. This definition implies that the two or more components were previously not in contact with one another, but through combining, the components come into contact with one another. Examples of components that may be combined include, but are not limited to, lactose, magnesium stearate, API, excipient preblend (i.e., lactose:MgSt mixture), and API preblend (i.e., API:MgSt mixture).

As used herein, the term “processing” or “processed” is used to refer to methods of particle size reduction and formulating including, but not limited to, blending, high-shear blending, milling, mechanofusion, mixing, and micronization methods such jet milling and ball milling. Examples of processing methods are described herein. In an embodiment, processing includes any significant handling of the preblends or the formulation. In an embodiment, significant handling of the preblends or the formulation includes blending the preblends or the formulation.

As used herein, the terms “resting,” “rested,” and “resting period” are used to refer to the methods of formulating whereby said formulation is idled and is thus not subjected to any further movement, processing, or any formulation procedures that would agitate the formulation during the specified period. It should be recognized that a purpose of the resting period is to allow any residual energy within the formulation to dissipate. In some embodiments, the residual energy within the formulation is static charge.

As used herein, the term “co-milling” or “co-milled” is used to refer to a range of powder processing methods used to break up agglomerates including, but not limited to, screen mills that can be various shapes including conical. In an embodiment, co-milling refers to screen milling.

As used herein, the terms “blend,” “blending,” and “blended” refer to mixing multiple components to obtain a formulation. The resulting blended formulation can be homogeneous. The term “blend” is also used herein to describe the product that results from blending. In this context, “blend” is synonymous with “formulation.”

As used herein, the term “high-shear blending” refers to combining or mixing multiple components to obtain a formulation wherein the method is conducted with a total input energy of 200-500 KJ/kg. Examples of high-shear blending methods include, but are not limited to, turbo rapid variable (TRV) blending.

As used herein, the term “content uniformity” refers to the uniformity of active pharmaceutical ingredient distributed throughout a blend. Content uniformity is expressed as the relative standard deviation (RSD) of the average of the sample assays.

As used herein, the terms “mass median aerodynamic diameter” and “MMAD” refer to the aerodynamic diameter of which half the particulate mass of the aerosol is contributed by particles with an aerodynamic diameter larger than the median and half by particles with an aerodynamic diameter smaller than the median. As used herein, the terms “MMAD” and “diameter” are used interchangeably.

As used herein, the term “fine particle mass” or “FPM” is used to refer to the mass of the metered dose made up of drug particles with an aerodynamic particle diameter of less than or equal to 5 μm.

As used herein, the term “fine particle fraction” or “FPF” refers to the fine particle mass as a percentage of the total amount of drug recovered from the cascade impactor, i.e. the total amount of drug recovered from the induction port to the micro-orifice collector (MOC).

As used herein, the term “target dose” is the amount of active pharmaceutical ingredient present in a single unit dose (e.g., the amount present in a single capsule or the amount present in a single blister). In some embodiments, the target dose is about 0.1-0.5 mg of Compound I. In some embodiments, the target dose is about 1.0-3.0 mg of Compound I. In some embodiments, the target dose is about 3.0-5.0 mg of Compound I. In some embodiments, the target dose is about 0.2 mg of Compound I. In some embodiments, the target dose is about 2.0 mg of Compound I. In some embodiments, the target dose is about 4.0 mg of Compound I.

As used herein, the term “delivered dose” is the amount of active pharmaceutical ingredient that is delivered to the subject per each actuation of the delivery device. Delivered dose is expressed as a percentage of the target dose of Compound I.

The present disclosure also includes salt forms of the compounds described herein. Examples of salts (or salt forms) include, but are not limited to, mineral or organic acid salts of basic residues such as amines, alkali or organic salts of acidic residues such as carboxylic acids, and the like. Lists of suitable salts are found in Remington's Pharmaceutical Sciences, 17th ed., Mack Publishing Company, Easton, Pa., 1985, p. 1418, the disclosure of which is hereby incorporated by reference in its entirety.

As used herein, the term “mild asthma” is asthma that is well controlled with as-needed reliever medication alone, or with low-intensity controller treatment such as low dose inhaled corticosteroid, leukotriene receptor antagonists, or chromones.

As used herein, the term “moderate asthma” is asthma that is well controlled with low dose to medium dose inhaled corticosteroid with or without a long-acting beta-agonist treatment (LABA) or other adjunctive controller treatments.

As used herein, the term “moderate to severe asthma” is asthma that requires medium to high-dose inhaled corticosteroid with or without long-acting beta-agonist or other adjunctive treatments to prevent it from becoming uncontrolled, or is asthma that remains uncontrolled despite this treatment.

It is to be understood that the term “asthma” is used to refer to any of “mild asthma,” “moderate asthma,” or “moderate to severe asthma.”

As used herein, the term “ICS naïve” is used to refer to patients who have never been treated with inhaled corticosteroids.

As used herein, the term “AUC_{0-∞}” refers to the total area under the plasma concentration-time curve extrapolated to an infinite time. Values of AUC_{0-∞} are reported in units of h·pg/mL or hours·picograms/milliliters.

As used herein, the term “AUC_{0-last non-zero}” refers to the total area under the plasma concentration-time curve from dosing (time 0) to the time of the last measurable (non-zero) concentration. Values of AUC_{0-last non-zero} are reported in units of h·pg/mL or hours·picograms/milliliters.

As used herein, the term “AUC_{over dosing interval}” refers to the total area under the plasma concentration-time curve over a 10-day dosing interval. Values of AUC_{over dosing interval} are reported in units of h·pg/mL or hours·picograms/milliliters.

As used herein, the term “AUC_{0-12 hours}” refers to the total area under the plasma concentration-time curve from dosing (time 0) to 12 hours post-dose (time 12). Values of AUC_{0-12 hours} are reported in units of h·pg/mL or hours·picograms/milliliters.

As used herein, the term “AUC_{0-24 hours}” refers to the total area under the plasma concentration-time curve from dosing (time 0) to 24 hours post-dose (time 24). Values of AUC_{0-24 hours} are reported in units of h·pg/mL or hours·picograms/milliliters.

As used herein, the term “C_{trough}” refers to the trough concentration of Compound I in the blood plasma prior to administration of a subsequent dose. Values of C_{trough} are reported in units of pg/mL or picograms/milliliters.

As used herein, the term “Cₘₐₓ” refers to the maximum concentration of Compound I in the blood plasma prior to administration of a subsequent dose. Values of Cₘₐₓ are reported in units of pg/mL or picograms/milliliters.

As used herein, the term “percent change” is used to quantify the change in a patient's eosinophil count upon administration of a formulation comprising Compound I. Percent change is calculated using the patient's baseline (screening) eosinophil count (Xᵢ) and the patient's eosinophil count upon administration of a formulation comprising Compound I (X_{f}) according to the following formula:

$$( \frac{{X}_{i} - {X}_{f}}{{X}_{i}} ) \times 100$$

It Is to be understood that a negative percent change indicates a reduction in the patient's eosinophil count upon administration of a formulation comprising Compound I.

### Formulations

As discussed in WO 2011/051452, (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile is a potent JAK2 and JAK3 inhibitor that has clinical potential in the treatment of respiratory indications such as asthma and COPD, as well as inflammatory processes associated with eosinophilic and non-eosinophilic inflammation. This compound has the following formula (see WO 2016/124464), and is also referred to herein as Compound I:

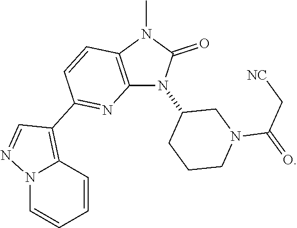

In some embodiments, the disclosure provides a pharmaceutical formulation comprising Compound I, or a pharmaceutically acceptable salt solvate, clathrate, or co-crystal thereof; one or more fillers; one or more glidants; and one or more lubricants.

The formulation can include Compound I in any suitable solid form, including amorphous, crystalline, or a combination thereof. For example, Compound I can exhibit any suitable crystalline form. Representative crystalline forms include one or more crystalline forms described in WO 2016/124464, which is incorporated herein in its entirety for all purposes.

In some embodiments, the disclosure provides a pharmaceutical formulation comprising Compound I:

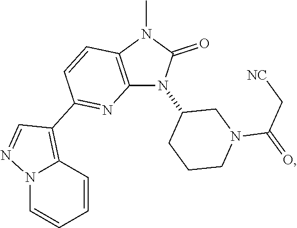

or a pharmaceutically acceptable salt thereof, lactose, and magnesium stearate.

In an embodiment, provided herein is the dry power pharmaceutical formulation formed by any of the methods disclosed herein.

In an aspect, provided herein is a pharmaceutical formulation comprising:
(a) (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile:

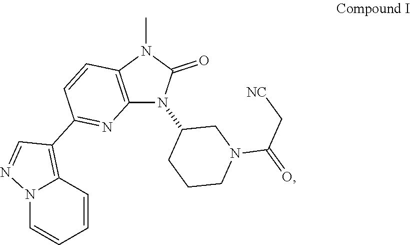

or a pharmaceutically acceptable salt thereof;
(b) magnesium stearate; and
(c) lactose.

In an embodiment, the formulation comprises:
(a) 20.0-25.0 weight percent Compound I, or a pharmaceutically acceptable salt thereof;
(b) 0.5-5.0 weight percent magnesium stearate; and
(c) 75.0-80.0 weight percent lactose.

In another embodiment, the formulation comprises:
(a) 0.3-20.0 weight percent Compound I, or a pharmaceutically acceptable salt thereof;
(b) 0.3-10.0 weight percent magnesium stearate; and
(c) 70.0-99.4 weight percent lactose.

In another embodiment, the formulation comprises:
(a) 0.4-13.0 weight percent Compound I, or a pharmaceutically acceptable salt thereof;
(b) 0.4-7.0 weight percent magnesium stearate; and
(c) 80.0-89.0 weight percent lactose.

In another embodiment, the formulation comprises:
(a) 0.5-11.0 weight percent Compound I, or a pharmaceutically acceptable salt thereof;
(b) 0.5-2.0 weight percent magnesium stearate; and
(c) 87.0-99.0 weight percent lactose.

In yet another embodiment, the formulation comprises:
(a) about 1.1 weight percent Compound I, or a pharmaceutically acceptable salt thereof;
(b) about 1.0 weight percent magnesium stearate; and
(c) about 97.9 weight percent lactose.

In still another embodiment, the formulation comprises:
(a) about 10.3 weight percent Compound I, or a pharmaceutically acceptable salt thereof;
(b) about 1.2 weight percent magnesium stearate; and
(c) about 88.5 weight percent lactose.

In an embodiment, the formulation comprises:
(a) 1.0-5.0 mg of Compound I, or a pharmaceutically acceptable salt thereof;
(b) 0.1-1.0 mg of magnesium stearate; and
(c) 15.0-37.0 mg of lactose.

In another embodiment, the formulation comprises:
(a) 0.1-1.0 mg of Compound I;
(b) 0.1-1.0 mg of magnesium stearate; and
(c) 18.0-20.0 mg of lactose.

In yet another embodiment, the formulation comprises:
(a) about 0.2 mg of Compound I, or a pharmaceutically acceptable salt thereof;
(b) about 0.2 mg of magnesium stearate; and
(c) about 19.6 mg of lactose.

In still another embodiment, the formulation comprises:
(a) about 2.1 mg of Compound I, or a pharmaceutically acceptable salt thereof;
(b) about 0.3 mg of magnesium stearate; and
(c) about 17.7 mg of lactose.

In an embodiment, the formulation comprises:
(a) about 4.1 mg of Compound I, or a pharmaceutically acceptable salt thereof;
(b) about 0.5 mg of magnesium stearate; and
(c) about 35.4 mg of lactose.

In an aspect, provided herein is a pharmaceutical formulation comprising: 100 weight percent (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile:

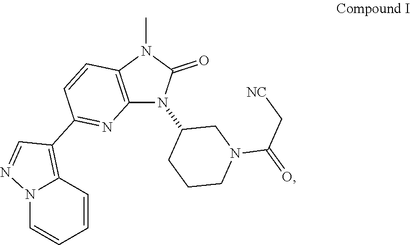

or a pharmaceutically acceptable salt thereof.

In another aspect, provided herein is a pharmaceutical formulation comprising:
(a) a first preblend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile:

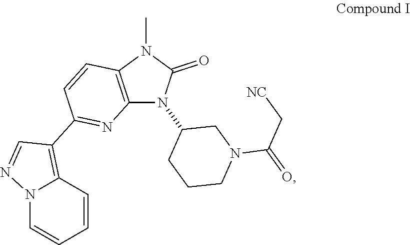

or a pharmaceutically acceptable salt thereof and magnesium stearate; and
(b) a second preblend of lactose and magnesium stearate.

In an embodiment, the first preblend is 1.0-15.0 weight percent magnesium stearate. In another embodiment, the first preblend is 1.0-11.0 weight percent magnesium stearate. In an embodiment, the first preblend is about 6.5 weight percent magnesium stearate.

In yet another embodiment, the first preblend is 1.0-11.0 weight percent magnesium stearate and the second preblend is 0.2-10.0 weight percent magnesium stearate. In still another embodiment, the first preblend is 1.0-11.0 weight percent magnesium stearate and the second preblend is 0.2-5.0 weight percent magnesium stearate. In an embodiment, the first preblend is 5.0-11.0 weight percent magnesium stearate and the second preblend is 0.2-10.0 weight percent magnesium stearate. In another embodiment, the first preblend is about 6.5 weight percent magnesium stearate and the second preblend is 0.2-10.0 weight percent magnesium stearate.

In an embodiment, the formulation is formulated as a bulk powder.

In another embodiment, the formulation is formulated as a capsule or a blister.

In an embodiment, the formulation is formulated as a capsule.

In yet another embodiment, the formulation comprises a bulk powder that is prepared without the use of plastic powder handling materials.

In still another embodiment, the formulation comprises a capsule that is prepared without the use of plastic powder handling materials.

In an embodiment, the formulation is prepared without plastic powder handling materials such as plastic spatulas, plastic scoops, or plastic transfer bags.

In another embodiment, the formulation is prepared without the use of a plastic transfer bag, such as a ChargeBag®.

In yet another embodiment, the formulation is prepared with stainless-steel powder handling materials.

In still another embodiment, the formulation is prepared with stainless-steel powder handling materials such as stainless-steel spatulas, stainless-steel scoops, stainless-steel funnels, or stainless-steel containers.

In some embodiments, the formulation is formulated as a capsule or a bulk powder. In some embodiments, the formulation is formulated as a capsule. In some embodiments, the formulation is formulated as a bulk powder.

In some embodiments, the formulation is formulated as a capsule or blister. In some embodiments the formulation is formulated as a blister.

In some embodiments, the formulation comprises about 0.5 wt % to about 11.0 wt % of Compound I, or a pharmaceutically acceptable salt thereof. In some embodiments, the formulation comprises about 0.9 wt % to about 1.3 wt % of Compound I, or a pharmaceutically acceptable salt thereof. In some embodiments, the formulation comprises about 9.5 wt % to about 10.8 wt % of Compound I, or a pharmaceutically acceptable salt thereof. In some embodiments, the formulation comprises about 1.1 wt % of Compound I, or a pharmaceutically acceptable salt thereof. In some embodiments, the formulation comprises about 10.3 wt % of Compound I, or a pharmaceutically acceptable salt thereof.

In some embodiments, the formulation comprises about 87.0 wt % to about 99.0 wt % lactose. In some embodiments, the formulation comprises about 88.0 wt % to about 89.0 wt % lactose. In some embodiments, the formulation comprises about 97.0 wt % to about 98.0 wt % lactose. In some embodiments, the formulation comprises about 88.5 wt % lactose. In some embodiments, the formulation comprises 97.9 wt % lactose.

In some embodiments, the formulation comprises about 0.5 wt % to about 2.0 wt % magnesium stearate. In some embodiments, the formulation comprises about 0.8 wt % to about 1.4 wt % magnesium stearate. In some embodiments, the formulation comprises about 1.0 wt % magnesium stearate. In some embodiments, the formulation comprises about 1.2 wt % magnesium stearate.

In some embodiments, the formulation comprises about 0.5 wt % to about 11 wt % of Compound I, or a pharmaceutically acceptable salt thereof, about 87.0 wt % to about 99.0 wt % lactose, and about 0.5 wt % to about 2.0 wt % magnesium stearate.

In some embodiments, the formulation comprises about 1.1 wt % of Compound I, or a pharmaceutically acceptable salt thereof, about 97.9 wt % lactose, and about 1.0 wt % magnesium stearate.

In some embodiments, the formulation comprises about 10.3 wt % of Compound I, or a pharmaceutically acceptable salt thereof, about 88.5 wt % lactose, and about 1.2 wt % magnesium stearate.

Any suitable form of lactose can be used in the formulations described herein. Crystalline lactose, amorphous lactose, and mixtures thereof are suitable for use in the formulations of the disclosure. In some embodiments, the lactose is a spray-dried mixture of crystalline and amorphous lactose.

Any suitable form of magnesium stearate can be used in the formulations described herein. Several types and grades of magnesium stearate are suitable for use in the disclosed formulations. Accordingly, magnesium stearate having varying specific surface areas and varying median particle sizes can be used in the formulations disclosed herein.

In some embodiments, the formulation is formulated for oral administration via an inhaler (e.g., a tablet or capsule). In some embodiments, the formulation is contained within capsule. In some embodiments, the formulation is contained within a blister. In some embodiments the formulation is formulated as a bulk powder.

In some embodiments, the formulation is formulated such that Compound I is present in an amount ranging from about 0.1 mg to about 4.2 mg. In some embodiments, Compound I is present in an amount of about 0.2 mg, about 2.0 mg, or about 4.0 mg. In some embodiments, Compound I is present in an amount of about 0.2 mg. In some embodiments, Compound I is present in an amount of about 2.0 mg. In some embodiments, Compound I is present in an amount of about 4.0 mg.

In some embodiments, the formulation is formulated such that Compound I is present in an amount of about 0.15-0.25 mg. Accordingly, in an embodiment, the formulation comprises about 0.2 mg of Compound I, about 19.6 mg lactose, and about 0.2 mg magnesium stearate. In some embodiments, the formulation is formulated as a 0.2 mg strength tablet or capsule. In some embodiments, the formulation is formulated as a 0.2 mg strength capsule.

In some embodiments, the formulation is formulated such that Compound I is present in an amount of about 1.5-2.5 mg. Accordingly, in some embodiments, the formulation comprises about 2.0 mg of Compound I, about 17.7 mg lactose, and about 0.3 mg magnesium stearate. In some embodiments, the formulation is formulated as a 2.0 mg strength tablet or capsule. In some embodiments, the formulation is formulated as a 2.0 mg strength capsule.

In some embodiments, the formulation is formulated such that Compound I is present in an amount of about 3.5-4.5 mg. Accordingly, in some embodiments, the formulation comprises about 4.0 mg of Compound I, about 35.4 mg lactose, and about 0.5 mg magnesium stearate. In some embodiments, the formulation is formulated as a 4.0 mg strength tablet or capsule. In some embodiments, the formulation is formulated as a 4.0 mg strength capsule.

Additional fillers or diluents for use in the formulations of the disclosure include fillers or diluents typically used in the formulation of pharmaceuticals. Examples of fillers or diluents for use in accordance with the disclosure include, but are not limited to, sugars such as lactose (e.g., anhydrous lactose, directly compressible anhydrous lactose, lactose monohydrate, modified lactose monohydrate), dextrose, glucose, sucrose, cellulose, starches and carbohydrate derivatives, polysaccharides (including dextrates and maltodextrin), polyols (including mannitol, xylitol, and sorbitol), cyclodextrins, calcium carbonates, magnesium carbonates, microcrystalline cellulose, combinations thereof, and the like. In some embodiments the filler or diluent is lactose, microcrystalline cellulose, or a combination thereof. In some embodiments the filler or diluent is trehalose.

The formulations of the disclosure can also comprise additional excipients, including surfactants, polymers, and binders. Surfactants suitable for use in the formulations of the disclosure include surfactants commonly used in the formulation of pharmaceuticals. Examples of surfactants include, but are not limited to, ionic- and nonionic surfactants or wetting agents commonly used in the formulation of pharmaceuticals, such as ethoxylated castor oil, polyglycolyzed glycerides, acetylated monoglycerides, sorbitan fatty acid esters, poloxamers, polyoxyethylene sorbitan fatty acid esters, polyoxyethylene derivatives, monoglycerides or ethoxylated derivatives thereof, diglycerides or polyoxyethylene derivatives thereof, sodium docusate, sodium laurylsulfate, cholic acid or derivatives thereof, lecithins, phospholipids, combinations thereof, and the like.

The safety and efficacy of solid drug products, as well as their robust performance, are ensured by meeting the specified values of critical quality attributes (CQAs). Among these, content uniformity (CU) in drug products is of primary importance. Content uniformity is defined as the measure of how evenly an API is distributed throughout a blend. A blend with superior content uniformity will have the same concentration of API throughout the entire blend, e.g., top, middle, and bottom. Content uniformity is measured by taking multiple samples from various locations throughout a blend and assaying each sample by HPLC or similar technique to determine the concentration of API in each sample. Content uniformity is expressed as the percent relative standard deviation (RSD) of the pooled samples from the concentration of the bulk blend.

In an embodiment, the content uniformity of the pharmaceutical formulation of Compound I has a percent relative standard deviation (RSD) less than or equal to about 5%. In another embodiment, the content uniformity of the pharmaceutical formulation of Compound I has an RSD less than or equal to about 4.0%. In yet another embodiment, the content uniformity of the pharmaceutical formulation of Compound I has an RSD less than or equal to about 3.0%.

In still another embodiment, the content uniformity of the pharmaceutical formulation of Compound I has an RSD less than or equal to about 2.0%. In an embodiment, the content uniformity of the pharmaceutical formulation of Compound I has an RSD less than or equal to about 1.5%. In another embodiment, the content uniformity of the pharmaceutical formulation of Compound I has an RSD less than or equal to about 1.0%. In yet another embodiment, the content uniformity of the pharmaceutical formulation of Compound I has an RSD less than or equal to about 0.5%.

Compound I is administered as a pharmaceutical formulation, typically with a pharmaceutically acceptable carrier or excipient. In one embodiment the disclosure relates to a pharmaceutical formulation of Compound I with one or more pharmaceutically acceptable excipients. Suitable compositions can be in the form of tablets, capsules, or inhalable compositions. In one embodiment the disclosure provides a capsule comprising Compound I. In one embodiment, the disclosure provides a capsule filled with a formulation comprising Compound I. In one embodiment, the disclosure provides a blister filled with a formulation comprising Compound I. In one embodiment the disclosure provides an inhalable composition comprising Compound I.

The formulation can be delivered via a dry powder inhaler (DPI) for the treatment of respiratory diseases. For administration by inhalation using a dry powder inhaler, Compound I can be administered as a dry powder formulation with one or more carrier substances. Suitable inhalation carriers are known in the art and in one embodiment include crystalline sugars such as monosaccharides or disaccharides. In one embodiment the carrier is lactose. Compound I can also be administered as a dry powder formulation without carrier substances.

Dry powder formulations of the disclosure may also have additional excipients such as force control agents. A force control agent is an additive which reduces the cohesion between the fine particles within the powder formulation. This promotes de-agglomeration when the powder is dispensed from the inhaler. Suitable force control agents such as magnesium stearate, are known in the art to enhance the stability of dry powder formulations. In one embodiment the force control agent is a metal stearate such as magnesium stearate.

The dry powder formulations of the disclosure can be administered using a unit dose dry powder inhaler or a multi-dose dry powder inhaler. In a non-limiting example, the dry powder formulations of the disclosure can be administered using various dry powder inhalers such as GyroHaler® or Miat® Monodose RS01 or a lever operated inhaler such as that disclosed in WO2009/092770. In another non-limiting example, the dry powder formulations of the disclosure can be administered using various open-inhale-close devices. In a further embodiment the disclosure provides a kit comprising an inhaler in combination with a formulation provided herein. In a further embodiment the disclosure provides a kit comprising a dry powder inhaler in combination with a pharmaceutical formulation provided herein. In an embodiment, the disclosure provides a kit comprising a Miat Monodose RS01 dry powder inhaler in combination with a pharmaceutical formulation provided herein.

In pulmonary administration, the size of the API particles is of great importance in determining the site of the absorption. The API particles must be very fine to be carried deep into the lungs, for example, having a mass median aerodynamic diameter of less than 10 μm. Particles having aerodynamic diameters greater than 10 μm are likely to impact the walls of the throat and generally do not reach the lungs. Particles having aerodynamic diameters in the range of 5 μm to 0.5 μm will generally be deposited in the respiratory bronchioles whereas smaller particles having aerodynamic diameters in the range of 2 to 0.05 μm are likely to be deposited in the alveoli.

In another embodiment, the formulation is suitable for aerosolized delivery to a subject.

In yet another embodiment, when aerosolized, the mass median aerodynamic diameter (MMAD) is less than or equal to about 5.0 μm (e.g., 0.5-5.0 μm, 1.0-4.0 μm, 1.5-3.0 μm, or 1.5-2.5 μm). In still another embodiment when aerosolized, the MMAD is about 1.9 μm. In another embodiment when aerosolized, the mass median aerodynamic diameter (MMAD) is about 1.8 μm.

Another critical parameter for inhaled dry powder formulations is the efficiency of dose delivery, which is measured by the fine particle fraction (FPF). Thus, the FPF provides an in vitro measure of the efficiency of the device/formulation in delivering the API to the lungs.

In an embodiment, when aerosolized, the fine particle fraction (FPF) of Compound I is greater than or equal to about 60% (e.g., 60-100%, 65-90%, 65-85%, 65-80%, 70-80%, or 70-78%). In another embodiment, when aerosolized, the FPF of Compound I is about 72.5-77.5%

The proportion of each metered dose consisting of drug particles of the correct size (MMAD) for deposition at the required site in the lungs also needs to be uniform (stable), i.e., within specified limits. This proportion is known as the fine particle mass (FPM) and represents the proportion of the metered dose made up of drug particles with an MMAD in the range of about 1 micron to about 5 microns. As well-known in the art, the FPM of a pMDI can be determined on a cascade impactor, such as the Next Generation Impactor (NCI). The FPM is the amount of the metered dose collected on particular stages of the cascade impactor (e.g. NGI).

In yet another embodiment, when a capsule containing about 1.0-3.0 mg of Compound I is aerosolized, the fine particle mass (FPM) of Compound I is 900-1800 μg. In still another embodiment, when a capsule containing about 1.0-3.0 mg of Compound I is aerosolized, the fine particle mass (FPM) of Compound I is 900-1500 μg. In yet another embodiment, when a capsule containing about 1.0-3.0 mg of Compound I is aerosolized, the fine particle mass (FPM) of Compound I is 900-1200 μg.

In still another embodiment, when a capsule containing about 3.0-5.0 mg of Compound I is aerosolized, the FPM of Compound I is 1750-2450 μg. In still another embodiment, when a capsule containing about 3.0-5.0 mg of Compound I is aerosolized, the FPM of Compound I is 1750-2350 μg. In an embodiment, when a capsule containing about 3.0-5.0 mg of Compound I is aerosolized, the FPM of Compound I is 1850-2250 μg. In another embodiment, when a capsule containing about 3.0-5.0 mg of Compound I is aerosolized, the FPM of Compound I is 1950-2250 μg.

In the case of dry powder inhalers (DPIs), it is important to balance the flow properties of the dry powder within the inhaler and the plume characteristics on inhalation. Coarse carrier particles, usually lactose, are used to aid the flow properties of the medicament, but it is important to ensure that the active ingredients separate from the coarse carrier on inhalation so that the fine particles of the active ingredients are entrained into the lungs. To provide an appropriate dose over the lifetime of the inhaler, it is important that this process occurs in a consistent manner. That is, inhalation devices must demonstrate a consistent delivered dose and fine particle mass from the first to the last dose.

In yet another embodiment, when aerosolized, the delivered dose of Compound I is at least about 80% of the target dose (e.g., 60-100%, 65-100%, 70-100%, and 75-90%). In still another embodiment, when aerosolized, the delivered dose of Compound I is 78-88% of the target dose.

In an embodiment, Compound I is a polymorph form having the following diffraction angles (2Theta) based on cupric Kα1:
at approximately 8.25°;
at approximately 13.25°;
at approximately 15.40°;
at approximately 17.65°; and
at approximately 25.39° (Form II).

### Polymorph Form II

In an embodiment of the formulations, methods of preparation, methods of treatment, and methods of FeNO reduction specified herein, (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I) is polymorph Form II having X-ray powder diffraction peak data according to the following table:

| 2θ (°) | Intensity | Rel. Intensity |
|---|---|---|
| 8.25 | 5933 | 100.0 |
| 10.80 | 1472 | 24.8 |
| 12.60 | 761 | 12.8 |
| 13.25 | 2310 | 38.9 |
| 15.40 | 3572 | 60.2 |
| 17.65 | 2549 | 43.0 |
| 18.35 | 420 | 7.1 |
| 18.80 | 1325 | 22.3 |
| 20.15 | 1081 | 18.2 |
| 20.70 | 1393 | 23.5 |
| 20.90 | 687 | 11.6 |
| 22.55 | 938 | 15.8 |
| 24.29 | 1301 | 21.9 |
| 24.74 | 480 | 8.1 |
| 25.39 | 2776 | 46.8 |
| 25.99 | 627 | 10.6 |
| 26.24 | 585 | 9.9 |
| 26.74 | 1481 | 25.0 |
| 27.34 | 451 | 7.6 |
| 32.99 | 542 | 9.1 |

In an embodiment, Compound I is a polymorph form having the following diffraction angles (2Theta) based on cupric Kα1:
at approximately 8.25°;
at approximately 13.25°;
at approximately 15.40°;
at approximately 17.65°; and
at approximately 25.39° (Form II).

This polymorph form and its corresponding methods of preparation are disclosed in U.S. Pat. No. 10,087,196, which is incorporated by reference herein in its entirety. Form II is further characterized by an endotherm onset at 239° C. as measured by differential scanning calorimetry.

### Methods of Use

Compound I has been shown to be a potent inhibitor of the JAK family of enzymes, specifically JAK1, JAK2, JAK3, and TYK2. For example, the biological activity of the compound can be found in WO 2011/051452, which is incorporated herein by reference in its entirety. Inhibition of the family of JAK enzymes could inhibit signaling of many key pro-inflammatory cytokines. Further, JAK inhibition represents an opportunity to interrupt inflammatory pathways implicated in the pathogenesis of multiple conditions including conditions associated with eosinophilic and non-eosinophilic inflammation that include respiratory diseases such as asthma, and chronic obstructive pulmonary disease (COPD). Thus, the formulations of the disclosure are useful in the treatment of conditions associated with eosinophilic and non-eosinophilic inflammation as defined herein, and respiratory diseases such as asthma and chronic obstructive pulmonary disease.

Compound I, and formulations comprising Compound I, are useful for the treatment of asthma and COPD, as well as other inflammatory conditions associated with eosinophilic inflammation and non-eosinophilic inflammation.

In an aspect, provided herein is a method of treating asthma, COPD, or an inflammatory condition associated with eosinophilic inflammation or non-eosinophilic inflammation in a subject in need thereof, wherein the method comprises administering to a subject a pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile, or a pharmaceutically acceptable salt thereof.

In an embodiment, the formulation further comprises lactose and magnesium stearate.

In an aspect, provided herein is a method of treating a respiratory condition comprising administering to a subject in need thereof a therapeutically-effective amount of a pharmaceutical formulation disclosed herein.

In some embodiments, the respiratory condition is asthma. In some embodiments, the respiratory condition is COPD. In some embodiments, the respiratory condition is associated with eosinophilic inflammation or non-eosinophilic inflammation. In some embodiments, the respiratory condition associated with eosinophilic inflammation or non-eosinophilic inflammation is asthma, COPD, nasal polyposis, rhinitis, pulmonary fibrotic disease, interstitial lung disease, or pulmonary hypertension. In some embodiments, the respiratory condition associated with eosinophilic inflammation or non-eosinophilic inflammation is asthma, nasal polyposis, rhinitis, pulmonary fibrotic disease, interstitial lung disease, or pulmonary hypertension. In some embodiments, the respiratory condition associated with eosinophilic inflammation or non-eosinophilic inflammation is nasal polyposis. In some embodiments, the respiratory condition associated with eosinophilic inflammation or non-eosinophilic inflammation is rhinitis. In some embodiments, the respiratory condition associated with eosinophilic inflammation or non-eosinophilic inflammation is pulmonary fibrotic disease. In some embodiments, the respiratory condition associated with eosinophilic inflammation or non-eosinophilic inflammation is interstitial lung disease. In some embodiments, the respiratory condition associated with eosinophilic inflammation or non-eosinophilic inflammation is pulmonary hypertension.

In some embodiments, the respiratory condition associated with eosinophilic inflammation or non-eosinophilic inflammation is mild, moderate, or severe asthma. In some embodiments, the formulation is administered once daily. In some embodiments, the formulation is administered twice daily. In some embodiments, the subject is a human. In some embodiments, the subject is ICS naïve.

In an embodiment, the method further comprises administering at least one of an inhaled corticosteroid, a long-acting β adrenoceptor agonist (LABA), a long-acting muscarinic antagonist (LAMA), or a short-acting beta agonist (SABA).

In another aspect, provided herein is a method of treating asthma, COPD, or an inflammatory condition associated with eosinophilic inflammation or non-eosinophilic inflammation in a subject in need thereof, wherein the method comprises administering to a subject a pharmaceutical formulation of the disclosure. In an embodiment, the inflammatory condition associated with eosinophilic inflammation or non-eosinophilic inflammation is eosinophilic asthma, nasal polyposis, rhinitis, pulmonary fibrotic disease, or interstitial lung disease.

In an embodiment, the inflammatory condition associated with eosinophilic inflammation or non-eosinophilic inflammation is eosinophilic asthma, nasal polyposis, rhinitis, pulmonary fibrotic disease, interstitial lung disease, or pulmonary hypertension.

In an aspect, provided herein is a method of treating asthma, COPD, or an inflammatory condition associated with eosinophilic inflammation or non-eosinophilic inflammation in a subject in need thereof, wherein the method comprises administering to a subject a pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile, or a pharmaceutically acceptable salt or co-crystal thereof.

In an aspect, provided herein is a method of treating asthma, COPD, or an inflammatory condition associated with eosinophilic inflammation or non-eosinophilic inflammation in a subject in need thereof, wherein the method comprises administering to a subject a pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile, or a pharmaceutically acceptable salt thereof.

In an embodiment, the pharmaceutical formulation further comprises lactose and magnesium stearate.

In another embodiment, the subject has mild asthma. In yet another embodiment, the subject has moderate asthma. In still another embodiment, the subject has moderate to severe asthma. In an embodiment, subject's moderate to severe asthma is characterized by a Th2-low phenotype. In another embodiment, the subject's moderate to severe asthma is characterized by a Th2-high phenotype.

In another embodiment, the subject is a human. In some embodiments, the formulation comprising Compound I is locally acting in the respiratory tract.

In yet another embodiment, administration of the formulation produces an AUC_{0-∞} between 30,000 h·pg/mL and 1,500,000 h·pg/mL. In still another embodiment, administration of the formulation produces an AUC_{0-∞} between 40,000 h·pg/mL and 600,000 h·pg/mL. In an embodiment, administration of the formulation produces an average AUC_{0-∞} of about 358,700 h·pg/mL. In another embodiment, the subject is administered about 4.0 mg of Compound I twice daily. In yet another embodiment, the subject has mild asthma.

In still another embodiment, administration of the formulation produces an AUC_{0-∞} between 132,000 h·pg/mL and 890,000 h·pg/mL. In an embodiment, administration of the formulation produces an average AUC_{0-∞} of about 439,000 h·pg/mL. In another embodiment, the subject is administered about 4.0 mg of Compound I twice daily. In yet another embodiment, the subject has moderate to severe asthma.

In still another embodiment, administration of the formulation produces an AUC_{0-∞} between 340,000 h·pg/mL and 1,400,000 h·pg/mL. In an embodiment, administration of the formulation produces an average AUC_{0-∞} of about 747,000 h·pg/mL. In another embodiment, the subject is administered about 8.0 mg of Compound I twice daily. In yet another embodiment, the subject has mild asthma.

In still another embodiment, administration of the formulation produces an AUC_{0-∞} between 2,000 h·pg/mL and 180,000 h·pg/mL. In an embodiment, administration of the formulation produces an AUC_{0-∞} between 7,000 h·pg/mL and 54,000 h·pg/mL. In another embodiment, administration of the formulation produces an average AUC_{0-∞} of about 28,000 h·pg/mL. In yet another embodiment, the subject is administered about 0.6 mg of Compound I once daily. In still another embodiment, the subject has mild asthma.

In an embodiment, administration of the formulation produces an AUC_{0-∞} between 49,000 h·pg/mL and 146,000 h·pg/mL. In another embodiment, administration of the formulation produces an average AUC_{0-∞} of about 89,000 h·pg/mL. In yet another embodiment, the subject is administered about 2.0 mg of Compound I once daily. In still another embodiment, the subject has mild asthma.

In yet another embodiment, administration of the formulation produces an AUC_{0-last non-zero} between 30,000 h·pg/mL and 1,500,000 h·pg/mL. In still another embodiment, administration of the formulation produces an AUC_{0-last non-zero} between 40,000 h·pg/mL and 600,000 h·pg/mL. In an embodiment, administration of the formulation produces an average AUC_{0-last non-zero} of about 355,000 h·pg/mL. In another embodiment, the subject is administered about 4.0 mg of Compound I twice daily. In yet another embodiment, the subject has mild asthma.

In still another embodiment, administration of the formulation produces an AUC_{0-last non-zero} between 132,000 h·pg/mL and 890,000 h·pg/mL. In an embodiment, administration of the formulation produces an average AUC_{0-last non-zero} of about 438,000 h·pg/mL. In another embodiment, the subject is administered about 4.0 mg of Compound I twice daily. In yet another embodiment, the subject has moderate to severe asthma.

In still another embodiment, administration of the formulation produces an AUC_{0-last non-zero} between 340,000 h·pg/mL and 1,400,000 h·pg/mL. In an embodiment, administration of the formulation produces an average AUC_{0-last non-zero} of about 745,000 h·pg/mL. In another embodiment, the subject is administered about 8.0 mg of Compound I twice daily. In yet another embodiment, the subject has mild asthma.

In still another embodiment, administration of the formulation produces an AUC_{0-last non-zero} between 2,000 h·pg/mL and 180,000 h·pg/mL. In an embodiment, administration of the formulation produces an AUC_{0-last non-zero} between 7,000 h·pg/mL and 50,000 h·pg/mL. In another embodiment, administration of the formulation produces an average AUC_{0-last non-zero} of about 27,000 h·pg/mL. In yet another embodiment, the subject is administered about 0.6 mg of Compound I once daily. In still another embodiment, the subject has mild asthma.

In an embodiment, administration of the formulation produces an AUC_{0-last non-zero} between 47,000 h·pg/mL and 146,000 h·pg/mL. In another embodiment, administration of the formulation produces an average AUC_{0-last non-zero} of about 87,000 h·pg/mL. In yet another embodiment, the subject is administered about 2.0 mg of Compound I once daily. In still another embodiment, the subject has mild asthma.

In yet another embodiment, administration of the formulation produces an AUC_{over dosing interval} between 20,000 h·pg/mL and 850,000 h·pg/mL. In still another embodiment, administration of the formulation produces an AUC_{over dosing interval} between 37,000 h·pg/mL and 375,000 h·pg/mL. In an embodiment, administration of the formulation produces an average AUC_{over dosing interval} of about 237,000 h·pg/mL. In another embodiment, the subject is administered about 4.0 mg of Compound I twice daily. In yet another embodiment, the subject has mild asthma.

In still another embodiment, administration of the formulation produces an AUC_{over dosing interval} between 103,000 h·pg/mL and 503,000 h·pg/mL. In an embodiment, administration of the formulation produces an average AUC_{over dosing interval} of about 278,000 h·pg/mL. In another embodiment, the subject is administered about 4.0 mg of Compound I twice daily. In yet another embodiment, the subject has moderate to severe asthma.

In still another embodiment, administration of the formulation produces an AUC_{over dosing interval} between 258,000 h·pg/mL and 832,000 h·pg/mL. In an embodiment, administration of the formulation produces an average AUC_{over dosing interval} of about 458,000 h·pg/mL. In another embodiment, the subject is administered about 8.0 mg of Compound I twice daily. In yet another embodiment, the subject has mild asthma.

In still another embodiment, administration of the formulation produces an AUC_{over dosing interval} between 2,000 h·pg/mL and 140,000 h·pg/mL. In an embodiment, administration of the formulation produces an AUC_{over dosing interval} between 7,000 h·pg/mL and 40,000 h·pg/mL. In another embodiment, administration of the formulation produces an average AUC_{over dosing interval} of about 23,000 h·pg/mL. In yet another embodiment, the subject is administered about 0.6 mg of Compound I once daily. In still another embodiment, the subject has mild asthma.

In an embodiment, administration of the formulation produces an AUC_{over dosing interval} between 40,000 h·pg/mL and 128,000 h·pg/mL. In another embodiment, administration of the formulation produces an average AUC_{over dosing interval} of about 76,000 h·pg/mL. In yet another embodiment, the subject is administered about 2.0 mg of Compound I once daily. In still another embodiment, the subject has mild asthma.

In yet another embodiment, administration of the formulation produces an AUC_{0-12 hours} between 20,000 h·pg/mL and 850,000 h·pg/mL. In still another embodiment, administration of the formulation produces an AUC_{0-12 hours} between 37,000 h·pg/mL and 375,000 h·pg/mL. In an embodiment, administration of the formulation produces an average AUC_{0-12 hours} of about 237,000 h·pg/mL. In another embodiment, the subject is administered about 4.0 mg of Compound I twice daily. In yet another embodiment, the subject has mild asthma.

In still another embodiment, administration of the formulation produces an AUC_{0-12 hours} between 258,000 h·pg/mL and 832,000 h·pg/mL. In an embodiment, administration of the formulation produces an average AUC_{0-12 hours} of about 458,000 h·pg/mL. In another embodiment, the subject is administered about 8.0 mg of Compound I twice daily. In yet another embodiment, the subject has mild asthma.

In still another embodiment, administration of the formulation produces an AUC_{0-12 hours} between 2,000 h·pg/mL and 100,000 h·pg/mL. In an embodiment, administration of the formulation produces an AUC_{0-12 hours} between 6,000 h·pg/mL and 25,000 h·pg/mL. In another embodiment, administration of the formulation produces an average AUC_{0-12 hours} of about 16,000 h·pg/mL. In yet another embodiment, the subject is administered about 0.6 mg of Compound I once daily. In still another embodiment, the subject has mild asthma.

In an embodiment, administration of the formulation produces an AUC_{0-12 hours} between 28,000 h·pg/mL and 90,000 h·pg/mL. In another embodiment, administration of the formulation produces an average AUC_{0-12 hours} of about 55,000 h·pg/mL. In yet another embodiment, the subject is administered about 2.0 mg of Compound I once daily. In still another embodiment, the subject has mild asthma.

In yet another embodiment, administration of the formulation produces an AUC_{0-24 hours} between 20,000 h·pg/mL and 1,200,000 h·pg/mL. In still another embodiment, administration of the formulation produces an AUC_{0-24 hours} between 42,000 h·pg/mL and 475,000 h·pg/mL. In an embodiment, administration of the formulation produces an average AUC_{0-24 hours} of about 299,000 h·pg/mL. In another embodiment, the subject is administered about 4.0 mg of Compound I twice daily. In yet another embodiment, the subject has mild asthma.

In still another embodiment, administration of the formulation produces an AUC_{0-24 hours} between 120,000 h·pg/mL and 702,000 h·pg/mL. In an embodiment, administration of the formulation produces an average AUC_{0-24 hours} of about 365,000 h·pg/mL. In another embodiment, the subject is administered about 4.0 mg of Compound I twice daily. In yet another embodiment, the subject has moderate to severe asthma.

In still another embodiment, administration of the formulation produces an AUC_{0-24 hours} between 308,000 h·pg/mL and 1,142,000 h·pg/mL. In an embodiment, administration of the formulation produces an average AUC_{0-24 hours} of about 612,000 h·pg/mL. In another embodiment, the subject is administered about 8.0 mg of Compound I twice daily. In yet another embodiment, the subject has mild asthma.

In still another embodiment, administration of the formulation produces an AUC_{0-24 hours} between 2,000 h·pg/mL and 135,000 h·pg/mL. In an embodiment, administration of the formulation produces an AUC_{0-24 hours} between 7,000 h·pg/mL and 37,000 h·pg/mL. In another embodiment, administration of the formulation produces an average AUC_{0-24 hours} of about 23,000 h·pg/mL. In yet another embodiment, the subject is administered about 0.6 mg of Compound I once daily. In still another embodiment, the subject has mild asthma.

In an embodiment, administration of the formulation produces an AUC_{0-24 hours} between 40,000 h·pg/mL and 128,000 h·pg/mL. In another embodiment, administration of the formulation produces an average AUC_{0-24 hours} of about 76,000 h·pg/mL. In yet another embodiment, the subject is administered about 2.0 mg of Compound I once daily. In still another embodiment, the subject has mild asthma.

In an embodiment, administration of the formulation produces a C_{trough} between 200 pg/mL and 50,000 pg/mL. In another embodiment, administration of the formulation produces a C_{trough} between 400 pg/mL and 17,000 pg/mL. In yet another embodiment, administration of the formulation produces an average C_{trough} of about 10,000 pg/mL. In still another embodiment, the subject is administered about 4.0 mg of Compound I twice daily. In an embodiment, the subject has mild asthma.

In another embodiment, administration of the formulation produces a C_{trough} between 1,000 pg/mL and 25,000 pg/mL. In yet another embodiment, administration of the formulation produces an average C_{trough} of about 11,000 pg/mL. In still another embodiment, the subject is administered about 4.0 mg of Compound I twice daily. In an embodiment, the subject has moderate to severe asthma.

In another embodiment, administration of the formulation produces a C_{trough} between 5,000 pg/mL and 42,000 pg/mL. In yet another embodiment, administration of the formulation produces an average C_{trough} of about 22,000 pg/mL. In still another embodiment, the subject is administered about 8.0 mg of Compound I twice daily. In an embodiment, the subject has mild asthma.

In another embodiment, administration of the formulation produces a C_{trough} between 20 pg/mL and 3,000 pg/mL. In yet another embodiment, administration of the formulation produces a C_{trough} between 30 pg/mL and 3,000 pg/mL. In still another embodiment, administration of the formulation produces an average C_{trough} of about 375 pg/mL. In an embodiment, the subject is administered about 0.6 mg of Compound I once daily. In another embodiment, the subject has mild asthma.

In yet another embodiment, administration of the formulation produces a C_{trough} between 400 pg/mL and 2,000 pg/mL. In still another embodiment, administration of the formulation produces an average C_{trough} of about 975 pg/mL. In an embodiment, the subject is administered about 2.0 mg of Compound I once daily. In another embodiment, the subject has mild asthma.

In an embodiment, administration of the formulation produces a maximum concentration (Cₘₐₓ) between 2,000 pg/mL and 110,000 pg/mL. In another embodiment, administration of the formulation produces a Cₘₐₓ between 6,000 pg/mL and 49,000 pg/mL. In yet another embodiment, administration of the formulation produces an average Cₘₐₓ of about 31,000 pg/mL. In still another embodiment, the subject is administered about 4.0 mg of Compound I twice daily. In an embodiment, the subject has mild asthma.

In another embodiment, administration of the formulation produces an Cₘₐₓ between 13,000 pg/mL and 65,000 pg/mL. In yet another embodiment, administration of the formulation produces an average Cₘₐₓ of about 38,000 pg/mL. In still another embodiment, the subject is administered about 4.0 mg of Compound I twice daily. In an embodiment, the subject has moderate to severe asthma.

In another embodiment, administration of the formulation produces a Cₘₐₓ between 43,000 pg/mL and 93,000 pg/mL. In yet another embodiment, administration of the formulation produces an average Cₘₐₓ of about 60,000 pg/mL. In still another embodiment, the subject is administered about 8.0 mg of Compound I twice daily. In an embodiment, the subject has mild asthma.

In another embodiment, administration of the formulation produces a Cₘₐₓ between 300 pg/mL and 15,000 pg/mL. In yet another embodiment, administration of the formulation produces a Cₘₐₓ between 900 pg/mL and 3,000 pg/mL. In still another embodiment, administration of the formulation produces an average Cₘₐₓ of about 2,000 pg/mL. In an embodiment, the subject is administered about 0.6 mg of Compound I once daily. In another embodiment, the subject has mild asthma.

In yet another embodiment, administration of the formulation produces a Cₘₐₓ between 3,000 pg/mL and 10,000 pg/mL. In still another embodiment, administration of the formulation produces an average Cₘₐₓ of about 6,000 pg/mL. In an embodiment, the subject is administered about 2.0 mg of Compound I once daily. In another embodiment, the subject has mild asthma.

In an embodiment, the above pharmacokinetic parameters can be measured 1, 2, 3, 4, 5, 6, 7, 8, 9, 10, 11, 12, 13, 14, 15, 16, 17, 18, 19, and/or 20 days after administration of the formulation.

In yet another embodiment, administration of the formulation produces a reduction in the induced sputum percent differential eosinophil count relative to pre-administration values. In still another embodiment, administration of the formulation produces a reduction in induced sputum percent differential eosinophil count between 1.5% and 20%. In another embodiment, administration of the formulation produces an average reduction in induced sputum percent differential eosinophil count of about 7%.

In an embodiment, the reduction in induced sputum percent differential eosinophil count is at least about 2%. In another embodiment, the reduction in induced sputum percent differential eosinophil count is at least about 4%. In yet another embodiment, the reduction in induced sputum percent differential eosinophil count is at least about 6%. In still another embodiment, the reduction in induced sputum percent differential eosinophil count is at least about 8%. In an embodiment, the reduction in induced sputum percent differential eosinophil count is at least about 10%. In another embodiment, the reduction in induced sputum percent differential eosinophil count is at least about 12%. In yet another embodiment, the reduction in induced sputum percent differential eosinophil count is at least about 14%. In still another embodiment, the reduction in induced sputum percent differential eosinophil count is at least about 16%. In an embodiment, the reduction in induced sputum percent differential eosinophil count is at least about 18%. In another embodiment, the reduction in induced sputum percent differential eosinophil count is at least about 20%.

In yet another embodiment, administration of the formulation produces a percent change in induced sputum percent differential eosinophil count, wherein the percent change is between −35% and −110%. In an embodiment, the average percent change in induced sputum percent differential eosinophil count is about −65%.

In another embodiment, the percent change in induced sputum percent differential eosinophil count is at least about −45%. In yet another embodiment, the percent change in induced sputum percent differential eosinophil count is at least about −50%. In still another embodiment, the percent change in induced sputum percent differential eosinophil count is at least about −55%. In an embodiment, the percent change in induced sputum percent differential eosinophil count is at least about −60%. In another embodiment, the percent change in induced sputum percent differential eosinophil count is at least about −65%. In yet another embodiment, the percent change in induced sputum percent differential eosinophil count is at least about −70%. In still another embodiment, the percent change in induced sputum percent differential eosinophil count is at least about −75%. In an embodiment, the percent change in induced sputum percent differential eosinophil count is at least about −80%. In another embodiment, the percent change in induced sputum percent differential eosinophil count is at least about −85%. In yet another embodiment, the percent change in induced sputum percent differential eosinophil count is at least about −90%. In still another embodiment, the percent change in induced sputum percent differential eosinophil count is at least about −95%. In yet another embodiment, the percent change in induced sputum percent differential eosinophil count is at least about −100%.

In yet another embodiment, the patient's induced sputum percent differential eosinophil count is reduced to less than about 3%. In still another embodiment, the above changes in induced sputum percent differential eosinophil count result from administration of about 4.0 mg of Compound I twice daily.

In yet another embodiment, administration of the formulation produces a reduction in the induced sputum absolute differential eosinophil count relative to pre-administration values. In still another embodiment, administration of the formulation produces a reduction in induced sputum absolute differential eosinophil count between 0.05×10⁶/g and 0.40×10⁶/g. In another embodiment, administration of the formulation produces an average reduction in induced sputum absolute differential eosinophil count of about 0.15×10⁶/g.

In an embodiment, the reduction in induced sputum absolute differential eosinophil count is at least about 0.05×10⁶/g. In another embodiment, the reduction in induced sputum absolute eosinophil count is at least about 0.1×10⁶/g. In yet another embodiment, the reduction in induced sputum absolute differential eosinophil count is at least about 0.15×10⁶/g. In still another embodiment, the reduction in induced sputum absolute differential eosinophil count is at least about 0.2×10⁶/g. In an embodiment, the reduction in induced sputum absolute differential eosinophil count is at least about 0.25×10⁶/g. In another embodiment, the reduction in induced sputum absolute differential eosinophil count is at least about 0.3×10⁶/g. In yet another embodiment, the reduction in induced sputum absolute differential eosinophil count is at least about 0.35×10⁶/g.

In still another embodiment, administration of the formulation produces a percent change in induced sputum absolute differential eosinophil count, wherein the percent change is between −60% and −110%. In an embodiment, the average percent change in induced sputum absolute differential eosinophil count is about −80%.

In another embodiment, the percent change in induced sputum absolute differential eosinophil count is at least about −65%. In an embodiment, the percent change in induced sputum absolute differential eosinophil count is at least about −70%. In yet another embodiment, the percent change in induced sputum absolute differential eosinophil count is at least about −75%. In still another embodiment, the percent change in induced sputum absolute differential eosinophil count is at least about −80%. In an embodiment, the percent change in induced sputum absolute differential eosinophil count is at least about −85%. In another embodiment, the percent change in induced sputum absolute differential eosinophil count is at least about −90%.

In yet another embodiment, the percent change in induced sputum absolute differential eosinophil count is at least about −95%. In still another embodiment, the percent change in induced sputum absolute differential eosinophil count is at least about −100%. In an embodiment, the percent change in induced sputum absolute differential eosinophil count is at least about −105%.

In another embodiment, the above changes in induced sputum absolute differential eosinophil count result from administration of about 4.0 mg of Compound I twice daily.

In yet another embodiment, administration of the formulation produces a reduction in the serum absolute differential eosinophil count relative to pre-administration values. In still another embodiment, administration of the formulation produces a reduction in serum absolute differential eosinophil count between 0.01×10⁹/L and 0.4×10⁹/L. In another embodiment, administration of the formulation produces an average reduction in serum absolute differential eosinophil count of about 0.15×10⁹/L.

In an embodiment, the reduction in serum absolute differential eosinophil count is at least about 0.03×10⁹/L. In another embodiment, the reduction in serum absolute differential eosinophil count is at least about 0.05×10⁹/L. In yet another embodiment, the reduction in serum absolute differential eosinophil count is at least about 0.1×10⁹/L. In still another embodiment, the reduction in serum absolute differential eosinophil count is at least about 0.15×10⁹/L. In an embodiment, the reduction in serum absolute differential eosinophil count is at least about 0.2×10⁹/L. In another embodiment, the reduction serum absolute differential eosinophil count is at least about 0.25×10⁹/L. In yet another embodiment, the reduction in serum absolute differential eosinophil count is at least about 0.3×10⁹/L. In still another embodiment, the reduction in serum absolute differential eosinophil count is at least about 0.35×10⁹/L. In an embodiment, the reduction in serum absolute differential eosinophil count is at least about 0.4×10⁹/L.

In yet another embodiment, administration of the formulation produces a percent change in serum absolute differential eosinophil count, wherein the percent change is between −2% and −80%. In an embodiment, the average percent change in serum absolute differential eosinophil count is about −29%.

In another embodiment, the percent change in serum absolute differential eosinophil count is at least about −4%. In yet another embodiment, the percent change in serum absolute differential eosinophil count is at least about −15%. In still another embodiment, the percent change in serum absolute differential eosinophil count is at least about −25%. In an embodiment, the percent change in serum absolute differential eosinophil count is at least about −32%. In another embodiment, the percent change in serum absolute differential eosinophil count is at least about −40%. In yet another embodiment, the percent change in serum absolute differential eosinophil count is at least about −43%. In still another embodiment, the percent change in serum absolute differential eosinophil count is at least about −45%. In an embodiment, the percent change in serum absolute differential eosinophil count is at least about −50%. In another embodiment, the percent change in serum absolute differential eosinophil count is at least about −54%. In yet another embodiment, the percent change in serum absolute differential eosinophil count is at least about −60%. In still another embodiment, the percent change in serum absolute differential eosinophil count is at least about −65%. In yet another embodiment, the percent change in serum absolute differential eosinophil count is at least about −70%. In still another embodiment, the percent change in serum absolute differential eosinophil count is at least about −74%. In an embodiment, the percent change in serum absolute differential eosinophil count is at least about −80%.

In another embodiment, the above changes in serum absolute differential eosinophil count result from administration of about 4.0 mg of Compound I twice daily.

Suitable doses for treating asthma and other respiratory disorders are expected to range from about 0.1 to about 100 mg/day of Compound I, including from about 0.2 to about 50 mg/day of active agent for an average 70 kg human. Suitable doses include, for example, about 0.001 mg/kg or 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 mg/kg.

In some embodiments, the subject is administered a formulation comprising Compound I at a dose of at least about 0.005 mg/kg. In some embodiments, the subject is administered a formulation comprising Compound I at a dose of from about 0.001 mg/kg to about 1 mg/kg. In some embodiments, the subject is administered a formulation comprising Compound I (e.g., a formulation comprising Compound I, MgSt, and lactose) at a dose of from about 0.1 mg/kg to about 1 mg/kg. In some embodiments, the subject is administered a formulation comprising Compound I at a dose of from about 0.01 mg/kg to about 0.1 mg/kg. In some embodiments, the subject is administered a formulation comprising Compound I (e.g., a formulation comprising Compound I, MgSt, and lactose) at a dose of 0.2 mg, 0.4 mg, 0.6 mg, 0.8 mg, 1.0 mg, 2.0 mg, 4.0 mg, 6.0 mg, 8.0 mg, 12.0 mg, 16.0 mg, 48.0 mg, or 68.0 mg. In some embodiments, the subject is administered a pharmaceutical formulation comprising Compound I (e.g., a pharmaceutical formulation comprising Compound I, MgSt, and lactose) at a dose of 0.2 mg, 0.6 mg, 2.0 mg, 4.0 mg, 6.0 mg, 8.0 mg, or 12.0 mg. In some embodiments, the subject is administered a pharmaceutical formulation comprising Compound I at a dose of about 0.2 mg. In some embodiments, the subject is administered a pharmaceutical formulation comprising Compound I at a dose of about 0.6 mg. In some embodiments, the subject is administered a pharmaceutical formulation comprising Compound I at a dose of about 2.0 mg. In some embodiments, the subject is administered a pharmaceutical formulation comprising Compound I at a dose of about 4.0 mg. In some embodiments, the subject is administered a pharmaceutical formulation comprising Compound I at a dose of about 6.0 mg. In some embodiments, the subject is administered a pharmaceutical formulation comprising Compound I at a dose of about 8.0 mg. In some embodiments, the subject is administered a pharmaceutical formulation comprising Compound I at a dose of about 12.0 mg.

In some embodiments, the subject is administered a pharmaceutical formulation comprising Compound I at any of the above doses once daily or twice daily. In some embodiments, the subject is administered a pharmaceutical formulation comprising Compound I at a dose of about 0.6 mg once daily. In some embodiments, the subject is administered a pharmaceutical formulation comprising Compound I at a dose of about 2.0 mg once daily. In some embodiments, the subject is administered a pharmaceutical formulation comprising Compound I at a dose of about 4.0 mg once daily. In some embodiments, the subject is administered a pharmaceutical formulation comprising Compound I at a dose of about 4.0 mg twice daily. In some embodiments, the subject is administered a pharmaceutical formulation comprising Compound I at a dose of about 8.0 mg once daily. In some embodiments, the subject is administered a pharmaceutical formulation comprising Compound I at a dose of about 8.0 mg twice daily. In some embodiments, the subject is administered a pharmaceutical formulation comprising Compound I at a dose of about 12.0 mg once daily.

In an aspect, provided herein is a method of treating a disease or disorder in a subject in need thereof, wherein the method comprises administering a therapeutically effective amount of any of the pharmaceutical formulations described herein in combination with at least one of a long-acting β adrenoceptor agonist, a long-acting muscarinic antagonist, a short-acting β adrenoceptor agonist, and an inhaled corticosteroid.

In some embodiments, the subject is administered a pharmaceutical formulation comprising Compound I (e.g., a pharmaceutical formulation comprising Compound I, MgSt, and lactose) in combination with at least one of a long-acting β adrenoceptor agonist, a long-acting muscarinic antagonist, or an inhaled corticosteroid. In some embodiments, the subject is administered a pharmaceutical formulation comprising Compound I as well as a long-acting β adrenoceptor agonist. In some embodiments, the subject is administered a pharmaceutical formulation comprising Compound I as well as a long-acting muscarinic antagonist. In some embodiments, the subject is administered a pharmaceutical formulation comprising Compound I as well as an inhaled corticosteroid. In some embodiments, the subject is administered a pharmaceutical formulation comprising Compound I as well as a short-acting β adrenoceptor agonist. In some embodiments, the subject is administered a pharmaceutical formulation comprising Compound I as well as a short-acting muscarinic antagonist.

In some embodiments, the subject is administered a pharmaceutical formulation comprising Compound I as well as an inhaled corticosteroid and a long-acting β adrenoceptor agonist. In some embodiments, the subject is administered a pharmaceutical formulation comprising Compound I as well as a long-acting β adrenoceptor agonist and a long-acting muscarinic antagonist. In some embodiments, the subject is administered a pharmaceutical formulation comprising Compound I as well as an inhaled corticosteroid, a long-acting β adrenoceptor agonist, and a long-acting muscarinic antagonist. In some embodiments, the subject is administered a pharmaceutical formulation comprising Compound I as well as a short-acting β adrenoceptor agonist and an inhaled corticosteroid. In some embodiments, the subject is administered a pharmaceutical formulation comprising Compound I as well as a short-acting β adrenoceptor agonist and a short-acting muscarinic antagonist. In some embodiments, the disease or disorder is asthma. In some embodiments, the disease or disorder is COPD.

### Methods of FeNO Reduction

In an aspect, provided herein is a method of reducing a subject's fractional exhaled nitric oxide (FeNO) concentration by administering to a subject in need thereof a pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile, or a pharmaceutically acceptable salt thereof.

In another aspect, provided herein is a method of reducing a subject's FeNO concentration, wherein the method comprises administering to a subject in need thereof a pharmaceutical formulation disclosed herein.

In another embodiment, the subject's FeNO concentration is reduced to at least about 35 parts per billion (ppb). In yet another embodiment, the subject's FeNO concentration is reduced to at least about 30 ppb. In still another embodiment, the subject's FeNO concentration is reduced to at least about 35 ppb. In an embodiment, the subject's FeNO concentration is reduced to at least about 20 ppb. In another embodiment, the subject's FeNO concentration is reduced to at least about 15 ppb. In yet another embodiment, the subject's FeNO concentration is reduced to at least about 10 ppb. In an embodiment, the subject's FeNO concentration is reduced relative to the subject's pre-administration FeNO concentration.

In another embodiment, the subject's FeNO concentration is reduced by at least about 20%. In yet another embodiment, the subject's FeNO concentration is reduced by at least about 30%. In still another embodiment, the subject's FeNO concentration is reduced by at least about 40%. In an embodiment, the subject's FeNO concentration is reduced by at least about 50%. In another embodiment, the subject's FeNO concentration is reduced by at least about 60%. In yet another embodiment, the subject's FeNO concentration is reduced by at least about 65%. In another embodiment, the subject's FeNO concentration is reduced by at least about 70%. In yet another embodiment, the subject's FeNO concentration is reduced by at least about 75%. In still another embodiment, the subject's FeNO concentration is reduced by at least about 80%. In an embodiment, the subject's FeNO concentration is reduced relative to both the subject's pre-administration FeNO concentration and the FeNO concentration upon placebo treatment.

In another embodiment, the formulation further comprises lactose and magnesium stearate. In still another embodiment, the formulation is administered in a single capsule.

Methods of Preparation Formulations comprising Compound I were found to contain large agglomerates as observed by visual inspection. In particular, when formulations were prepared by combining lactose:MgSt preblends with API:MgSt preblends without an intermediate co-milling step, there was a higher content uniformity variability (i.e., a reduced level of homogeneity) and lower drug assay. In contrast, formulations prepared with an intermediate co-milling step, i.e., co-milling the lactose:MgSt preblend and the API:MgSt preblend prior to blending, the content uniformity improved. This suggests that an additional co-milling step is necessary to mix the components of the formulation to produce formulations comprising Compound I that are suitable for clinical studies.

In an aspect, provided herein is a method for the preparation of a dry powder pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I), wherein the method comprises:
(a) co-milling (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile with magnesium stearate to form a first preblend;
(b) co-milling magnesium stearate and lactose to form a second preblend; and
(c) blending the first and second preblends together to form the formulation.

In some embodiments, a portion of the second preblend is used to coat the co-mill prior to co-milling the first preblend. In some embodiments, a portion of the second preblend is combined with the first preblend prior to co-milling the first preblend.

In some embodiments, the method comprises co-milling a preblend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate, wherein the preblend is 4.0-14.0 weight percent magnesium stearate.

In some embodiments, the method comprises co-milling a preblend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate, wherein the preblend is 4.0-8.0 weight percent magnesium stearate.

In some embodiments, the method comprises co-milling a preblend of lactose and magnesium stearate, wherein the preblend is 0.4-1.1 weight percent magnesium stearate.

In some embodiments, the method comprises co-milling a preblend of lactose and magnesium stearate, wherein the preblend is 0.8-1.1 weight percent magnesium stearate.

In some embodiments, the method comprises conducting step (c) by high-shear blending.

In some embodiments, the method comprises conducting step (c) by TRV blending.

In some embodiments, the formulation is contained within a capsule or a blister.

In some embodiments, the formulation is contained within a capsule.

In addition, formulations comprising Compound I were also found to exhibit static adhesion to plastic powder handling materials. Specifically, when the formulations were transferred out of plastic transfer bags, significant amounts of the bulk powder adhered to the inner walls of the transfer bag as observed through visual inspection. The static adhesion resulted in formulations with significant variability across drug content measurements and suboptimal content uniformity. By replacing the plastic transfer bag with a clean GMP stainless-steel container and incorporating a resting period (e.g., 10-120 hr) into the formulation method prior to further substantial handling or processing, the static adhesion was reduced. As such, formulations prepared with clean GMP stainless-steel containers and resting periods prior to testing resulted in consistent and increased drug content and improved content uniformity.

In another aspect, provided herein is a method for the preparation of a dry powder pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I), wherein the method comprises:
(a) combining (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile with magnesium stearate to form a first preblend;
(b) combining magnesium stearate and lactose to form a second preblend;
(c) blending the first and second preblends together to form the formulation; and
(d) resting the formulation.

In yet another aspect, provided herein is a method for the preparation of a dry powder pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I), wherein the method comprises:
(a) combining (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile with magnesium stearate to form a first preblend;
(b) combining magnesium stearate and lactose to form a second preblend;
(c) blending the first and second preblends together to form the formulation; and
(d) resting the formulation for 10-120 hours.

In some embodiments, the method comprises a resting step (d) of 15-65 hours.

In some embodiments, after the resting period, the formulation is further handled. Further handling can include, but is not limited to, additional processing, additional formulation steps, or any other activities necessary to prepare the formulation for administration to the subject.

In some embodiments, the method comprises forming a preblend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate, wherein the preblend is 4.0-14.0 weight percent magnesium stearate.

In some embodiments, the method comprises forming a preblend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate, wherein the preblend is 4.0-8.0 weight percent magnesium stearate.

In some embodiments, the method comprises forming a preblend of lactose and magnesium stearate, wherein the preblend is 0.4-1.1 weight percent magnesium stearate.

In some embodiments, the method comprises forming a preblend of lactose and magnesium stearate, wherein the preblend is 0.8-1.1 weight percent magnesium stearate.

In an embodiment, the first preblend is formed in a container, and the second preblend is formed in a separate container prior to these preblends being combined. The preblends can be formed in separate containers prior to step (c).

In some embodiments, the method comprises conducting step (c) by high-shear blending.

In some embodiments, the method comprises conducting step (c) by TRV blending.

In some embodiments, the method comprises the additional step of:
(e) filling a capsule with the formulation.

In some embodiments, the method comprises the additional step of:
(e) filling a blister with the formulation.

In some embodiments, the method is carried out without the use of plastic powder handling materials.

In some embodiments, the method is carried out without the use of plastic powder handling materials including plastic spatulas, plastic scoops, and plastic transfer bags.

In some embodiments, the method is carried out without the use of plastic transfer bags including ChargeBag®.

In some embodiments, the method is carried out with stainless-steel powder handling materials.

In some embodiments, the method is carried out with stainless-steel powder handling materials including stainless-steel spatulas, stainless-steel scoops, stainless-steel funnels, and stainless-steel containers.

The formulations of the disclosure can be prepared by combining any of the concepts mentioned above, i.e., replacing plastic powder handling materials with stainless-steel powder handling materials to reduce static adhesion, incorporating a resting period (10-120 hr) to reduce static adhesion, and incorporating an intermediate co-milling step to reduce agglomerate size.

In an aspect, provided herein is a method for the preparation of a dry powder pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile, wherein the method comprises:
(a) combining (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I), or a pharmaceutically acceptable salt thereof, with magnesium stearate to form a first preblend;
(b) combining magnesium stearate and lactose to form a second preblend;
(c) blending the first and second preblends together to form the formulation; and
(d) resting the formulation for 10-120 hours.

In some embodiments, the resting step (d) is 15-65 hours.

The formulation is rested prior to any further significant handling or processing. Further significant handling includes, but is not limited to, additional formulation steps or any other activities necessary to prepare the formulation for administration to the subject.

In some embodiments, the method comprises co-milling the first and second preblends prior to step (c).

In some embodiments, a portion of the second preblend is used to coat the co-mill prior to co-milling the first and second preblends.

In some embodiments, the method comprises co-milling a preblend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate, wherein the preblend is 4.0-14.0 weight percent magnesium stearate.

In some embodiments, the method comprises co-milling a preblend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate, wherein the preblend is 4.0-8.0 weight percent magnesium stearate.

In some embodiments, the method comprises co-milling a preblend of lactose and magnesium stearate, wherein the preblend is 0.4-1.1 weight percent magnesium stearate.

In some embodiments, the method comprises co-milling a preblend of lactose and magnesium stearate, wherein the preblend is 0.8-1.1 weight percent magnesium stearate.

In an embodiment, the first preblend is formed in a container, and the second preblend is formed in a separate container prior to these preblends being combined. The preblends can be formed in separate containers prior to step (c).

In some embodiments, the method comprises conducting step (c) by high-shear blending.

In some embodiments, the method comprises conducting step (c) by TRV blending.

In some embodiments, the method comprises the additional step of:
(e) filling a capsule with the formulation.

In some embodiments, the method comprises the additional step of:
(e) filling a blister with the formulation.

In another aspect, provided herein is a method for the preparation of a dry powder pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I), wherein the method comprises:
(a) co-milling (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I) or a pharmaceutically acceptable salt thereof, with magnesium stearate to form a first preblend;
(b) co-milling magnesium stearate and lactose to form a second preblend; and
(c) blending the first and second preblends together to form the formulation.

In some embodiments, a portion of the second preblend is used to coat the co-mill prior to co-milling the first preblend. In some embodiments, a portion of the second preblend is combined with the first preblend prior to co-milling the first preblend.

In some embodiments, the method comprises co-milling a pre-blend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate, wherein the preblend is 4.0-14.0 weight percent magnesium stearate.

In some embodiments, the method comprises co-milling a pre-blend of (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate, wherein the preblend is 4.0-8.0 weight percent magnesium stearate.

In some embodiments, the method comprises co-milling a preblend of lactose and magnesium stearate, wherein the preblend is 0.4-1.1 weight percent magnesium stearate.

In some embodiments, the method comprises co-milling a preblend of lactose and magnesium stearate, wherein the preblend is 0.8-1.1 weight percent magnesium stearate.

In some embodiments, the method comprises the additional step of:
(d) resting the formulation for 10-120 hours.

In some embodiments, the method comprises the additional step of:
(d) resting the formulation for 15-65 hours.

In some embodiments, the method comprises conducting step (c) by high-shear blending.

In some embodiments, the method comprises conducting step (c) by TRV blending.

In some embodiments, the method comprises the additional step of:
(e) filling a capsule with the formulation.

In some embodiments, the method comprises the additional step of:
(e) filling a blister with the formulation.

In yet another aspect, provided herein is a method for the preparation of a dry powder pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I), wherein the method comprises:
(a) combining (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I), or a pharmaceutically acceptable salt thereof with magnesium stearate to form a first preblend;
(b) combining magnesium stearate and lactose to form a second preblend;
(c) combining the first preblend with the second preblend;
(d) co-milling the product formed in step (c); and
(e) blending the product formed in step (d) to form the formulation.

In some embodiments, a portion of the second preblend is co-milled prior to step (c).

In some embodiments, the first preblend is 4.0-14.0 weight percent magnesium stearate.

In some embodiments, first preblend is 4.0-8.0 weight percent magnesium stearate.

In some embodiments, the second preblend is 0.4-1.1 weight percent magnesium stearate.

In some embodiments, the second preblend is 0.8-1.1 weight percent magnesium stearate.

In some embodiments, the method comprises the additional step of:
(f) resting the formulation for 10-120 hours.

In some embodiments, the method comprises the additional step of:
(f) resting the formulation for 15-65 hours.

In some embodiments, the method comprises conducting step (e) by high-shear blending.

In some embodiments, the method comprises conducting step (e) by TRV blending.

In some embodiments, the method comprises the additional step of:
(g) filling a capsule with the formulation.

In some embodiments, the method comprises the additional step of:
(h) filling a blister with the formulation.

In an embodiment, after the resting period, the formulation is further handled. Further handling can include, but is not limited to, additional formulation steps or any other activities necessary to prepare the formulation for administration to the subject.

In some embodiments, the method is carried out without the use of plastic powder handling materials.

In some embodiments, the method is carried out without the use of plastic powder handling materials including plastic spatulas, plastic scoops, and plastic transfer bags.

In some embodiments, the method is carried out without the use of plastic transfer bags including ChargeBag®.

In some embodiments, the method is carried out with stainless-steel powder handling materials.

In some embodiments, the method is carried out with stainless-steel powder handling materials including stainless-steel spatulas, stainless-steel scoops, stainless-steel funnels, and stainless-steel containers.

The drug particles or particles of pharmaceutically active material (also referred to herein as “active particles”) in the formulations of the disclosure must aerosolise into an ultra-fine aerosol so that they can be transported to the appropriate target area within the lung (vide supra). Typically, for lung deposition, the active particles have a diameter of less than 10 μm, frequently 0.1 to 7 μm, 0.1 to 5 μm, or 0.5 to 5 μm.

For formulations to reach the deep lung or the blood stream via inhalation, the active agent in the formulation must be in the form of very fine particles, for example, having a mass median aerodynamic diameter (MMAD) of less than 10 μm. It is well established that particles having an MMAD of greater than 10 μm are likely to impact on the walls of the throat and generally do not reach the lung. Particles having an MMAD in the region of 5 to 2 μm will generally be deposited in the respiratory bronchioles whereas particles having an MMAD in the range of 3 to 0.05 μm are likely to be deposited in the respiratory bronchioles and alveoli allowing benefit on small airways inflammation found in respiratory diseases that include asthma and COPD.

When dry powders are produced using conventional processes, the active particles will vary in size, and often this variation can be considerable. This can make it difficult to ensure that a high enough proportion of the active particles are of the appropriate size for administration to the correct site. It is therefore desirable to have a dry powder formulation wherein the size distribution of the active particles is as narrow as possible. This will improve dose efficiency and reproducibility. Fine particles, that is, those with an MMAD of less than 5 μm and smaller, tend to be increasingly thermodynamically unstable as their surface area to volume ratio increases, which provides an increasing surface free energy with this decreasing particle size, and consequently increases the tendency of particles to agglomerate. Agglomeration of fine particles and adherence of such particles to the walls of the inhaler can result in the fine particles leaving the inhaler as large, stable agglomerates, or being unable to leave the inhaler and remaining adhered to the interior of the inhaler, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung.

These micron to submicron particle sizes required for deep lung delivery lead to the problem that the respirable active particles tend to be highly cohesive, which means they generally exhibit poor flowability and poor aerosolization.

In order to improve the properties of powder formulations, and in particular to improve the flowability and dispersibility of the formulation, dry powder formulations often include additive materials which are intended to reduce the cohesion between the fine particles in the dry powder formulation. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, where upon the particles are expelled from the device and inhaled.

The compositions described herein can include an additive material (for example in the form of distinct particles of a size comparable to that of the fine active particles).

The additive material can be an anti-adherent material and that will reduce the cohesion between particles and will also prevent fine particles becoming attached to surfaces within the inhaler device. Advantageously, the additive material is an anti-friction agent or glidant and will give the powder formulation better flow properties in the inhaler. The additive materials used in this way may not necessarily be usually referred to as anti-adherents or anti friction agents, but they will have the effect of decreasing the cohesion between the particles or improving the flow of the powder. As such, the additive materials are sometimes referred to as force control agents (FCAs) and they usually lead to better dose reproducibility and higher fine particle fractions (FPFs).

Therefore, an additive material or FCA, as used herein, is a material whose presence on the surface of a particle can modify the adhesive and cohesive surface forces experienced by that particle, in the presence of other particles and in relation to the surfaces that the particles are exposed to. In general, its function is to reduce both the adhesive and cohesive forces.

The reduced tendency of the particles to bond strongly, either to each other or to the device itself, not only reduces powder cohesion and adhesion, but can also promote better flow characteristics. This leads to improvements in the dose reproducibility because it reduces the variation in the amount of powder metered out for each dose and improves the release of the powder from the device. It also increases the likelihood that the active material which does leave the device will reach the lower lung of the subject.

The use of additive materials in this manner is disclosed in, for example, WO 1996/023485 and WO 1997/003649.

It is also known that intensive co-milling of micronized drug particles with additive material may be carried out in order to produce composite particles. This co-milling can improve dispersibility, as disclosed in the earlier patent application published as WO 2002/043701. In addition, the earlier application published as WO 2002/000197 discloses the intensive co-milling of fine particles of excipient material with additive material, to create composite excipient particles to which fine active particles and, optionally, coarse carrier particles may be added. This has also been shown to improve dispersibility.

At least two types of methods can be used herein in the context of processing active and additive particles. First, there is the compressive type process, such as mechanofusion and the cyclomix and related methods such as the hybridizer or the nobilta. As the name suggests, mechanofusion is a dry coating process designed to mechanically fuse a first material onto a second material. The first material is generally smaller and/or softer than the second. The principles behind the mechanofusion and cyclomix processes are distinct from those of alternative milling techniques in that they have a particular interaction between an inner element and a vessel wall, and in that they are based on providing energy by a controlled and substantial compressive force.

The fine active particles and the additive particles are fed into the mechanofusion driven vessel (such as a mechanofusion system (Hosokawa Micron Ltd)), where they are subject to a centrifugal force which presses them against the vessel inner wall. The inner wall and a curved inner element together form a gap or nip in which the particles are pressed together. The powder is compressed between the fixed clearance of the drum wall and a curved inner element with high relative speed between drum and element. As a result, the particles experience very high shear forces and very strong compressive stresses as they are trapped between the inner drum wall and the inner element (which has a greater curvature than the inner drum wall). The particles are pressed against each other with enough energy to locally heat and soften, break, distort, flatten and wrap the additive particles around the active particles to form coatings. The energy is generally sufficient to break up agglomerates and some degree of size reduction of both components may occur. While the coating may not be complete, the deagglomeration of the particles during the process ensures that the coating may be substantially complete, covering the majority of the surfaces of the particles.

These mechanofusion and cyclomix processes apply a high enough degree of force to separate the individual particles of active material and to break up tightly bound agglomerates of the active particles such that effective mixing and effective application of the additive material to the surfaces of those particles is achieved.

An especially desirable aspect of the described processing methods is that the additive material becomes deformed during mechanofusion and may be smeared over or fused to the surfaces of the active particles. However, in practice, this compression process produces little or no size reduction of the drug particles, especially where they are already in a micronized form (i.e., <10 μm). The only physical change which may be observed is a plastic deformation of the particles to a rounder shape.

Additional processing techniques include those described in R. Pfeffer et al. “Synthesis of engineered particulates with tailored properties using dg particle coating,” Powder Technology 117 (2001) 40-67. These include processes using a mechanofusion machine, a hybidizer machine, a theta composer, magnetically assisted impaction processes and rotating fluidized bed coaters. Cyclomix methods may also be used.

The technique can be employed to apply the required mechanical energy and involves the compression of a mixture of particles of the dispersing agent and particles of the pharmaceutically active agent in a nip formed between two portions of the machine, as is the case in the mechanofusion and cyclomix devices. Some processing methods are described below.

### Mechanofusion:

This dry coating process is designed to mechanically fuse a first material onto a second material. The first material is generally smaller and/or softer than the second. The mechanofusion and cyclomix working principles are distinct from alternative processing techniques in having a particular interaction between inner element and vessel wall and are based on providing energy by a controlled and substantial compressive force.

The fine active particles and the particles of dispersing agent are fed into the mechanofusion driven vessel, where they are subject to a centrifugal force and are pressed against the vessel inner wall. The powder is compressed between the fixed clearance of the drum wall and a curved inner element with high relative speed between drum and element. The inner wall and the curved element together form a gap or nip in which the particles are pressed together. As a result, the particles experience very high shear forces and very strong compressive stresses as they are trapped between the inner drum wall and the inner element (which has a greater curvature than the inner drum wall). The particles violently collide against each other with enough energy to locally heat and soften, break, distort, flatten and wrap the particles of dispersing agent around the core particle to form a coating. The energy is generally Sufficient to break up agglomerates and some degree of size reduction of both components may occur. Embedding and fusion of additive particles of dispersing agent onto the active particles may occur, and may be facilitated by the relative differences in hardness (and optionally size) of the two components. Either the outer vessel or the inner element may rotate to provide the relative movement. The gap between these surfaces is relatively small, and is typically less than 10 mm and can be less than 5 mm or less than 3 mm. This gap is fixed, and consequently leads to a better control of the compressive energy than is provided in some other forms of mill such as ball and media mills. Also, in general, no impaction of milling media surfaces is present so that wear and consequently contamination are minimized. The speed of rotation may be in the range of 200 to 10,000 rpm. A scraper may also be present to break up any caked material building up on the vessel surface. This is particularly advantageous when using fine cohesive starting materials. The local temperature may be controlled by use of a heating/cooling hacked built into the drum vessel walls. The powder may be re-circulated through the vessel.

### Cyclomix Method (Hosokawa Microm):

The cyclomix comprises a stationary conical vessel with a fast rotating shaft with paddles that move close to the wall. Due to the high rotational speed of the paddles, the powder is propelled towards the wall, and as a result the mixture experiences very high shear forces and compressive stresses between wall and paddle. Such effects are similar to those in mechanofusion as described above and may be sufficient to locally heat and soften, to break, distort, flatten and wrap the particles of dispersing agent around the active particles to form a coating. The energy is sufficient to break up agglomerates and some degree of size reduction of both components may also occur depending on the conditions and upon the size and nature of the particles.

### Hybridizer Method:

This is a dry process that can be described as a product embedding or filming of one powder onto another. The fine active particles and fine or ultra fine particles of dispersing agent are fed into a conventional high shear mixer pre-mix system to form an ordered mixture. This powder is then fed into the hybridizer. The powder is subjected to ultra-high speed impact, compression and shear as it is impacted by blades on a high speed rotor inside a stator vessel, and is re-circulated within the vessel. The active and additive particles collide with each other. Typical speeds of rotation are in the range of 5,000 to 20,000 rpm. The relatively soft fine particles of dispersing agent experience sufficient impact force to soften, break, distort, flatten and wrap around the active particle to form a coating. There may also be some degree of embedding into the surface of the active particles.

The second of the types of processes mentioned in the prior art is the impact milling processes. Such impact milling is involved, for example, in ball milling, jet milling and the use of a homogenizer.

Ball milling is a milling method used in many of the prior art processing methods. Centrifugal and planetary ball milling can be employed.

Jet mills are capable of reducing solids to particle sizes in the low-micron to submicron range. The grinding energy is created by gas streams from horizontal grinding air nozzles. Particles in the fluidized bed created by the gas streams are accelerated towards the center of the mill, colliding with slower moving particles. The gas streams and the particles carried in them create a violent turbulence and, as the particles collide with one another, they are pulverized.

High pressure homogenizers involve a fluid containing the particles being forced through a valve at high pressure, producing conditions of high shear and turbulence. Suitable homogenizers include EmulsiFlex high pressure homogenizers which are capable of pressures up to 4000 bar, Niro Soavi high pressure homogenizers (capable of pressures up to 2000 bar) and Micro fluidics Microfluidizers (maximum pressure 2750 bar).

Milling may, alternatively, involve a high energy media mill or an agitator bead mill, for example, the Netzsch high energy media mill, or the DYNO-mill (Willy A. Bachofen AG, Switzerland).

All of these processes create high-energy impacts between media and particles or between particles. In practice, while these processes are good at making very small particles, it has been found that the ball mill, jet mill and the homogenizer were not as effective in producing dispersion improvements in resultant drug powders as the compressive type processes. It is believed that the impact processes discussed above are not as effective in producing a coating of additive material on each particle as the compressive type processes.

For the purposes of this disclosure, all forms of co-milling are encompassed, including methods that are similar or related to all of those methods described above. For example, methods similar to mechanofusion are encompassed, such as those utilizing one or more very high-shear rotors (i.e., 2000 to 50000 rpm) with blades or other elements sweeping the internal surfaces of the vessels with small gaps between wall and blade (i.e., 0.1 mm to 20 mm). Conventional methods comprising co-milling active material with additive materials (as described in WO 2002/043701) are also encompassed. These methods result in composite active particles comprising ultra-fine active particles with an amount of the additive material on their surfaces.

In the past, jet milling has been considered less attractive for micronizing active and additive particles in the preparation of powder formulations to be dispensed using passive devices. The collisions between the particles in a jet mill are somewhat uncontrolled and those skilled in the art, therefore, considered it unlikely that this technique would be able to provide the desired deposition of a coating of additive material on the surface of the active particles.

Moreover, it was believed that, unlike the situation with compressive type processes such as mechanofusion and cyclomixing, segregation of the powder constituents occurred in jet mills, such that the finer particles, that were believed to be the most effective, could escape from the process. In contrast, it could be clearly envisaged how techniques such as mechanofusion would result in the desired coating.

Jet milling has been shown to be an attractive process for micronizing active and additive particles, especially for preparing powder formulations that are to be used in active devices (see the disclosure in the earlier patent application published as WO 2004/001628, incorporated herein by reference in its entirety). In another embodiment, the additive material may be in the form of particles adhering to the surfaces of the active and carrier particles. The additive material can become fused to the surfaces of the active and carrier particles.

Carrier particles may be of any acceptable inert excipient material or combination of materials. For example, carrier particles frequently used in the prior art may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously, the carrier particles comprise a polyol. In particular, the carrier particles may be particles of crystalline sugar, for example mannitol, dextrose, or lactose. In some embodiments, the carrier particles are trehalose particles. The carrier particles can be composed of lactose.

The additive may comprise a metal stearate, or a derivative thereof, for example, sodium stearyl fumarate or sodium stearyl lactylate. Advantageously, it comprises a metal stearate, for example, zinc stearate, magnesium stearate, calcium stearate, sodium stearate or lithium stearate. The additive material can comprise magnesium stearate, for example vegetable magnesium stearate, or any form of commercially available metal stearate, which may be of vegetable or animal origin and may also contain other fatty acid components such as palmitates or oleates.

### Articles of Manufacture and Kits

The formulations described herein can be prepared, placed in an appropriate container, and labeled for treatment of an indicated condition. Accordingly, there also is contemplated an article of manufacture, such as a container comprising a pharmaceutical formulation described herein and a label containing instructions for use of the composition.

In some embodiments, the article of manufacture is a container comprising a pharmaceutical formulation described herein. In some embodiments of the articles of manufacture described herein, the formulation is contained within a capsule.

Kits are also contemplated. For example, a kit can comprise a pharmaceutical formulation of the present disclosure and a package insert containing instructions for use of the composition in treatment of a medical condition. In some embodiments, a kit may comprise multiple formulations as described herein, each comprising a therapeutically effective amount of Compound I, and instructions for their administration to a human in need thereof.

### EQUIVALENTS AND SCOPE

While various disclosure embodiments have been particularly shown and described in the present disclosure, it will be understood by those skilled in the art that various changes in form and details may be made without departing from the spirit and scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

Compound I, (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile, can be prepared according to the procedures outlined in WO 2011/051452 (see Example 7i) and WO 2016/124464 (see Form II, Example 2.2), both of which are incorporated herein by reference in their entireties. Any and all references to Compound I herein in the examples refer to polymorph Form II.

Abbreviations as used herein have respective meanings as follows:

| | |
|---|---|
| API | Active pharmaceutical ingredient |
| AE | Adverse event(s) |
| APSD | Aerodynamic particle size distribution |
| AUC_{0-∞} | Area under the plasma concentration-time |
| | curve extrapolated to an infinite time |
| AUC_{0-last non-zero} | Area under the plasma concentration-time |
| | curve from dosing (time 0) to the time of the |
| | last measurable (non-zero) concentration |
| AUC_{0-12 hours} | Area under the plasma concentration-time |
| | curve from dosing (time 0) to 12 hours post- |
| | dose (time 12) |
| AUC_{0-24 hours} | Area under the plasma concentration-time |
| | curve over the last 24 h dosing interval |
| C_{trough} | Trough concentration of Compound I in the |
| | blood plasma prior to administration of a |
| | subsequent dose |
| Cₘₐₓ | Maximum concentration of Compound I in |
| | the blood plasma prior to administration of |
| | a subsequent dose |
| CSS | Croscarmellose sodium |
| F | Female |
| FeNO | Fractional exhaled nitric oxide |
| GMP | Good manufacturing practice |
| g | Gram(s) |
| h or hr | Hour(s) |
| HPMC | Hydroxypropyl methylcellulose |
| ICS | Inhaled corticosteroid(s) |
| kg | Kilogram(s) |
| L | Liter(s) |
| L-HPC | Low-substituted hydroxypropyl cellulose |
| MGST or MgSt | Magnesium Stearate |
| M | Male |
| MoS | Margin of safety |
| MTD | Maximum tolerated dose |
| MCC | Microcrystalline Cellulose |
| mcg | Microgram(s) |
| mg | Milligram(s) |
| MMAD | Median mass aerodynamic diameter |
| μm or mcm | Micrometer(s) |
| MOC | Micro-orifice collector |
| mir | Minute(s) |
| mL | Milliliter(s) |
| mm | Millimeter(s) |
| ng | Nanogram(s) |
| NGI | Next generation impactor |
| NHP | Nonhuman primate |
| NOAEL | No observed adverse effect level |
| OD/QD | Once daily |
| PK | Pharmacokinetics |
| pg | Picogram(s) |
| TK | Toxicokinetics |
| tox | Toxicology |
| TEAE | Treatment emergent adverse event(s) |
| TRV | Turbo rapid variable |
| BID | Twice daily |
| RSD | Relative standard deviation |
| rpm | Revolutions per minute |
| Std Dev | Standard deviation |
| vol | Volume |
| wt | Weight |
| wt % | Weight Percent |
| μL | Microliter(s) |

### General Procedure for Drug Content Determination

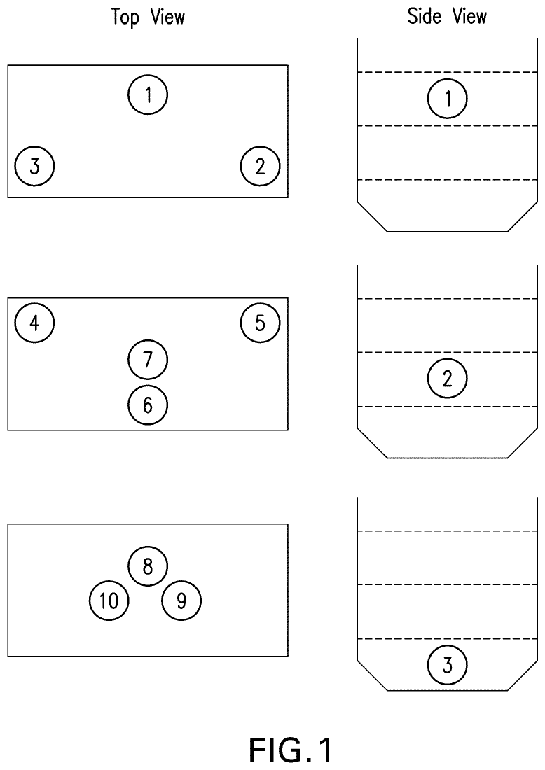

All drug content measurements were performed using location 7 as depicted in FIG. 1.

### 1 wt % Compound I Formulation

Approximately 300 mg of the formulation containing Compound I was transferred to a vial. The sample was transferred to a 500 mL volumetric flask and diluted to approximately 6 mcg/mL. Approximately 200 mL of the diluent was then sonicated for 10 minutes. The content of Compound I in the diluted sample was then determined by HPLC.

### 10 wt % Compound I Formulation

Approximately 400 mg of the formulation containing Compound I was transferred to a vial. The sample was transferred to a 500 mL volumetric flask and diluted to approximately 80 mcg/mL. Approximately 200 mL of the diluent was then sonicated for 10 minutes. Approximately 20 ml of the sonicated diluent was transferred to a 100 mL volumetric flask and diluted to an approximate concentration of 16 mcg/mL. The content of Compound I in the diluted sample was then determined by HPLC.

### General Procedure for Content Uniformity Determination

All content uniformity measurements were performed using locations 1-10 as depicted in FIG. 1.

### 1 wt % Compound I Formulation

Approximately 60 mg of the formulation containing Compound I was transferred to a vial. The sample was transferred to a 100 mL volumetric flask and diluted to approximately 6 mcg/mL. Approximately 50 mL of the diluent was then sonicated for 10-20 minutes. The content uniformity of Compound I in the diluted sample was then determined by HPLC. This process was repeated for a total of 10 samples.

### 10 wt % Compound I Formulation

Approximately 120 mg of the formulation containing Compound I was transferred to a vial. The sample was transferred to a 500 mL volumetric flask and diluted to approximately 24 mcg/mL. Approximately 250 mL of the diluent was then sonicated for 10-20 minutes. The content uniformity of Compound I in the diluted sample was then determined by HPLC. This process was repeated for a total of 10 samples.

### General Procedure for APSD, MMAD, FPM, and FPF Determination

### Cup Coating Agent Preparation

Cup coating agent was prepared by mixing 150 mL of a mixture of Brij® 35, ethanol, and glycerol with 850 ml of ethanol.

### NGI Procedure

The cup coating agent was prepared, and the NGI was assembled. The collection cups were then coated with cup coating agent and then proceeded to dry. A PALL® A/E 76 mm filter was placed into the back-up filter and attached to the NGI. Approximately 15 mL of diluent was added into the pre-separator insert. The flow rate was set to 90 L/min and the capsule was pierced. One dose was discharged into the NGI by initiating the time-controlled solenoid valve for 2.70 seconds. Sample diluent was added (10 mL for 0.2 mg and 2.0 mg doses and 20 mL for 4.0 mg doses), and the collection cups were agitated on the NGI Gentle Rocker for 10 minutes. The induction port was added 40 mL of sample diluent and was shaken for 1 minute. The pre-separator was added 50 mL of sample diluent and was shaken for 1 minute. The back-up filter was transferred to a crystallizing dish with 20 mL of sample diluent and was then sonicated for 5 minutes. The capsule from each determination was then transferred to a 20 mL volumetric flask and sample diluent was added. The suspension was then shaken and sonicated until fully dissolved. The resulting solution equilibrated under ambient conditions and was then made up to volume. Each Monodose RS01 was placed into an individual plastic bag and 20 mL of recovery solution was added. The bag was shaken by hand for 1 minute and filtered prior to HPLC analysis.

### General Procedure for Compound I Delivered Dose Determination

A Castellated fixed volume dosage unit sampling apparatus (DUSA) was assembled and attached to a backup filter. PALL® A/E 47 mm filter papers were used in the DUSA and PALL® A/E 76 mm filter were used in the backup filter. The solenoid timer was then equipped and the flow rate was adjusted to 90 L/min. The pre-sample weight of the device was recorded and the inhaler was activated. The dose was discharged twice into the DUSA by initiating the timer controlled solenoid valve. The post-sample weight of the device was then recorded and the exhaust bung was inserted into the exhaust port. Sample diluent (50 mL) was introduced into the DUSA through the mouthpiece adaptor, and the DUSA was placed on orbital shaker for 5 minutes at 250 shakes per minute. The capsule is dissolved in diluent and drug retained on the device is recovered by washing with diluent. Dilutions were performed with 4 mg capsules. All samples were filtered through a disposable 0.2 μm PTFE syringeless filter with polypropylene housing into HPLC vials and analyzed via HPLC.

### Example 1: Formulations Comprising Compound I

### Blend 1 (2 kg Scale)

A 1 wt % magnesium stearate excipient preblend, i.e., lactose:MgSt, was prepared by combining lactose, 10% fines (3961.3 g) with magnesium stearate (38.8 g). An 88.0 wt % (12.0 wt % magnesium stearate) magnesium stearate API preblend, i.e., API:MgSt, was prepared by combining Compound I (35.4 g) with magnesium stearate (4.8 g). Approximately 1977.1 g of the lactose:MgSt preblend was combined with 22.8 g of the API:MgSt preblend. The resulting mixture was then homogenized in a TRV blender at 500 rpm for 14 minutes to afford a 1.0 wt % API bulk powder formulation (Blend 1). The blend 1 bulk powder was transferred to a plastic ChargeBag® prior to testing.

Content uniformity and drug content as determined for the Blend 1 bulk powder are provided in Table 1.

TABLE 1

| Test | Acceptance Criteria | Blend 1 |
|---|---|---|
| Content Uniformity | ≤5.0% RSD | 13.4 |
| Drug Content | 0.85-1.15 | 0.8 |
| | wt % | |

### Blend 1a

To investigate the effects of static charge on the drug content and content uniformity of the bulk powder, Blend 1 was reprocessed in a stainless-steel container according to the below procedure. During the transfer to a stainless-steel container, large agglomerates of Blend 1 were observed, suggesting that an additional co-milling step is necessary in the manufacture of formulations of Compound I.

The Blend 1 bulk powder was transferred to a clean, sealed GMP stainless-steel container (160 mm×160 mm) and reprocessed via co-milling at 1000 rpm. The mixture was then homogenized via TRV blending at 1000 rpm for 7 minutes to afford the formulation (Blend 1a). Content uniformity and drug content as determined for Blend 1a are provided in Table 2. These data show that an improved content uniformity is achieved with a co-milling step in the manufacture of Blend 1a. However, the drug content of Blend 1a remained low.

TABLE 2

| Test | Acceptance Criteria | Blend 1a |
|---|---|---|
| Content Uniformity | ≤5.0% RSD | 4.9 |
| Drug Content | 0.85-1.15 | 0.7 |
| | wt % | |

### Example 2: Low-Strength Formulation of Compound I (0.2 mg)

To further probe the effects of static charge within formulations of Compound 1, a resting period was introduced into the manufacturing process according to the below procedures. Additionally, the magnesium stearate content in the API:MgSt preblend was reduced to investigate potential effects on the formulations of Compound I.

Blend 2a (with Co-Milling) (0.4 kg Scale)

A 0.9 wt % magnesium stearate excipient preblend, i.e., lactose:MgSt, was prepared by combining lactose, 10% fines (3962.4 g) with magnesium stearate (37.6 g). A 6.5 wt % magnesium stearate API preblend, i.e., API:MgSt, was prepared by combining Compound I (69.9 g) with magnesium stearate (4.9 g). In a subsequent step, a U5 Comil® with a 457 mcm sieve was used to process 395.1 g of the lactose:MgSt preblend and 4.8 g of the API:MgSt preblend at 1000 rpm into a 1 L TRV bowl. The resulting mixture was then homogenized in a TRV blender at 1209 rpm for 4 minutes to afford a 1 wt % API bulk powder formulation (Blend 2a). The Blend 2a bulk powder was transferred to a clean, sealed GMP stainless-steel container (160 mm×160 mm) which followed with a resting period (20 hours, 40 hours, or 60 hours) prior to capsule filling. Capsules were hand-filled with 20 mg of the Blend 2a bulk powder for a 0.2 mg target dose.

The resulting composition of the 0.2 mg product is shown in Table 3.

TABLE 3

| | Amount | Composition |
|---|---|---|
| Component | (mg) | (wt %) |
| Compound I | 0.2 | 1.1 |
| Magnesium Stearate | 0.2 | 1.0 |
| Lactose | 19.6 | 97.9 |

Content uniformity and drug content as determined for the Blend 2a bulk powder along with aerodynamic particle size distribution as determined for the Blend 2a capsules is provided in Table 4.

Blend 2b (without Co-Milling) (0.4 kg Scale)

A 0.9 wt % magnesium stearate excipient preblend, i.e., lactose:MgSt, was prepared by combining lactose, 10% fines (3962.4 g) with magnesium stearate (37.6 g). A 6.5 wt % magnesium stearate API preblend, i.e., API:MgSt, was prepared by combining Compound I (69.9 g) with magnesium stearate (4.9 g). Approximately 395.3 g of the lactose:MgSt preblend was combined with 4.8 g of the API:MgSt preblend. The resulting mixture was then homogenized in a TRV blender at 1209 rpm for 4 minutes to afford a 1 wt % API bulk powder formulation (Blend 2b). The Blend 2b bulk powder was transferred to a clean, sealed GMP stainless-steel container (160 mm×160 mm) which followed with a resting period (20 hours, 40 hours, or 60 hours) prior to capsule filling. Capsules were hand-filled with 20 mg of the Blend 2b bulk powder for a 0.2 mg target dose.

Content uniformity and drug content as determined for the Blend 2b bulk powder along with aerodynamic particle size distribution as determined for the Blend 2b capsules is provided in Table 4. A comparison of the Blend 2a (comil) and Blend 2b (no comil) standard deviations from the aerodynamic particle size distribution individual stage profiles is provided in Table 5.

TABLE 4

| | | Blend 2a | | | Blend 2b | | |
|---|---|---|---|---|---|---|---|
| | Acceptance | (Comil) | | | (No Comil) | | |
| Test | Criteria | 20 Hr | 40 Hr | 60 Hr | 20 Hr | 40 Hr | 60 Hr |
| Content | ≤5.0% RSD | 1.4 | 1.4 | 2.0 | 4.2 | 4.6 | 1.2 |
| Uniformity | | | | | | | |
| Drug | 0.85-1.15 wt % | 1.07 | 1.07 | 1.07 | 1.09 | 1.07 | 1.07 |
| Content | | | | | | | |
| APSD | Mean Total | 94.1 | 99.1 | 101.4 | 104.1 | 98.7 | 96.9 |
| | Recovery as % | | | | | | |
| | of label claim | | | | | | |
| | Mean Fine | 62.9 | 58.4 | 60.5 | 58.1 | 52.4 | 59.3 |
| | Particle | | | | | | |
| | Fraction (%) | | | | | | |

As can be seen from Table 4, reduced content uniformity variability of the Blend 2b bulk powder was observed after a rest period of 60 hours compared to rest periods of 20 and 40 hours as noted by a decrease in RSD. Further, a higher drug delivery efficiency as inferred from mean FPF was observed when a co-milling step was used in the manufacture of formulations comprising Compound I. Also, compared to Table 1, the data in Table 4 suggests that the use of stainless steel equipment, co-milling, rest periods, and a lower concentration of magnesium stearate in the API:MgSt preblend results in a higher drug content for formulations comprising Compound I.

TABLE 5

| | 20 Hr | | 40 Hr | | 60 Hr | |
|---|---|---|---|---|---|---|
| | Blend 2a | Blend 2b | Blend 2a | Blend 2b | Blend 2a | Blend 2b |
| | (Comil) | (No Comil) | (Comil) | (No Comil) | (Comil) | (No Comil) |
| Impactor stage | Std dev | Std dev | Std dev | Std dev | Std dev | Std dev |
| Device | 3.7 | 2.8 | 11.6 | 12.6 | 2.3 | 3.4 |
| Capsule | 0.8 | 2.6 | 0.8 | 10.4 | 1.7 | 5.5 |
| Induction Port | 2.4 | 3.7 | 3.1 | 9.3 | 2.3 | 1.5 |
| Pre-Separator | 1.3 | 1.6 | 1.7 | 8.5 | 1.3 | 3.0 |
| Stage 1 | 1.1 | 0.1 | 0.6 | 2.3 | 0.5 | 1.1 |
| Stage 2 | 2.9 | 0.6 | 0.5 | 10.1 | 1.0 | 1.3 |
| Stage 3 | 1.6 | 1.3 | 0.5 | 11.2 | 1.0 | 1.1 |
| Stage 4 | 2.9 | 3.5 | 1.0 | 4.9 | 0.9 | 1.2 |
| Stage 5 | 1.3 | 1.2 | 0.5 | 1.0 | 0.7 | 0.5 |
| Stage 6 | 0.6 | 0.8 | 0.4 | 0.5 | 0.4 | 0.4 |
| Stage 7 | 0.6 | 0.7 | 0.2 | 0.6 | 0.3 | 0.3 |
| MOC | 0.4 | 0.4 | 0.2 | 0.2 | 0.1 | 0.2 |

The results from Table 5 show a decrease in variability of the standard deviations from the APSD individual stage profiles after a rest period of 60 hours compared to rest periods of 20 or 40 hours for Blend 2b. Table 5 also shows that a decrease in variability was observed after rest periods of 40 and 60 hours compared to a rest period of 20 hours for Blend 2a. Further, a decrease in variability was observed for Blend 2a compared to Blend 2b for rest periods of 40 and 60 hours.

### Example 3: High-Strength Formulations of Compound I (2.0 mg and 4.0 mg)

### Blend 3 (0.4 kg Scale)

A 0.6 wt % magnesium stearate excipient preblend, i.e., lactose:MgSt, was prepared by combining lactose, 6% fines (3976.8 g) with magnesium stearate (23.3 g). A 6.5 wt % magnesium stearate API preblend, i.e., API:MgSt, was prepared by combining Compound I (69.9 g) with magnesium stearate (4.9 g). In a subsequent step, a U5 Comil® with a 457 mcm sieve was used to process 356.0 g of the lactose:MgSt preblend and 44.0 g of the API:MgSt preblend at 1000 rpm into a 1 L TRV bowl to produce a mixture. The resulting mixture was then homogenized in a TRV blender at 2392 rpm for 4 minutes to afford a 10 wt % API bulk powder formulation (Blend 3). The Blend 3 bulk powder was then transferred to a clean, sealed GMP stainless-steel container (160 mm×160 mm) which followed with a resting period (20 hours or 60 hours) prior to capsule filling. Size 3 HPMC capsules were hand-filled with 20 mg of the Blend 3 bulk powder for a 2.0 mg target dose and 40 mg of the Blend 3 bulk powder for a 4.0 mg target dose. The resulting composition of the 2.0 mg and 4.0 mg products is shown in Table 6.

TABLE 6

| | Amount (mg) | Amount (mg) | Composition |
|---|---|---|---|
| Component | 20 mg Capsule | 40 mg Capsule | (wt %) |
| Compound I | 2.1 | 4.1 | 10.3 |
| Magnesium Stearate | 0.3 | 0.5 | 1.2 |
| Lactose | 17.7 | 35.4 | 88.5 |

Content uniformity, drug content, and aerodynamic particle size distribution as determined for the bulk powder, 20 mg capsule, and 40 mg capsule are provided in Table 7. Standard deviations from the aerodynamic particle size distribution individual stage profiles for the Blend 3 capsules are provided in Table 8.

TABLE 7

| | | | | 20 mg Filled Capsules | | 40 mg Filled Capsules | |
|---|---|---|---|---|---|---|---|
| | Acceptance | Bulk Powder | | (2.0 mg Target Dose) | | (4.0 mg Target Dose) | |
| Test | Criteria | 20 Hr | 60 Hr | 20 Hr | 60 Hr | 20 Hr | 60 Hr |
| Content | ≤5.0% RSD | 1.6 | 0.2 | | | | |
| Uniformity | | | | | | | |
| Drug | 8.5-11.5 wt % | 9.6 | 9.5 | | | | |
| Content | | | | | | | |
| APSD | Mean Total | | | 89.7 | 85.4 | 86.1 | 81.9 |
| | Recovery as % | | | | | | |
| | of label claim | | | | | | |
| | Mean Fine | | | 46.2 | 56.7 | 49.8 | 52.9 |
| | Particle | | | | | | |
| | Fraction (%) | | | | | | |

Improved homogeneity of the Blend 3 bulk powder was observed after a rest period of 60 hours compared to a rest period of 20 hours as noted by a decrease in RSD. In addition, an increase in the mean fine particle fraction was observed after a rest period of 60 hours compared to a rest period of 20 hours for both the 20 mg (2.0 mg of Compound I) and 40 mg (4.0 mg of Compound I) capsules filled with Blend 3. In addition, the improved drug content relative to Blend 1 and Blend 1a (Table 1 and Table 2) further supports that the use of stainless steel equipment, co-milling, and rest periods are necessary to produce suitable formulations of Compound I, and that a lower concentration of magnesium stearate in the API:MgSt preblend is necessary to produce formulations comprising Compound I, magnesium stearate, and lactose that are suitable for clinical studies.

Thus, the improved homogeneity of Blend 3 as inferred from content uniformity (Table 7) and the improved drug content of Blend 3 resulted in clinical investigations of formulations comprising Compound I, lactose, and magnesium stearate. Stated alternatively, the methods of preparation of the dry powder pharmaceutical formulations of Compound I, as well as the formulations of Compound I, lactose, and magnesium stearate disclosed herein resulted in formulations comprising Compound I suitable for clinical trial investigation. Prior to the instant discovery, both the lack of homogeneity of Blend 1 as inferred from the poor content uniformity and the low drug content (Table 1) precluded clinical studies of prior formulations comprising Compound I, lactose, and magnesium stearate.

TABLE 8

| | Blend 3 Std dev | | | |
|---|---|---|---|---|
| | 20 mg Capsules | | 40 mg Capsules | |
| | (2.0 mg Target Dose) | | (4.0 mg Target Dose) | |
| Impactor stage | 20 Hr | 60 Hr | 20 Hr | 60 Hr |
| Device | 162.1 | 43.2 | 31.3 | 3.4 |
| Capsule | 35.6 | 14.9 | 13.1 | 15.1 |
| Induction Port | 34.0 | 10.8 | 63.2 | 16.7 |
| Pre-Separator | 150.0 | 16.7 | 68.1 | 30.4 |
| Stage 1 | 2.4 | 6.2 | 15.9 | 3.9 |
| Stage 2 | 47.4 | 29.9 | 9.1 | 2.1 |
| Stage 3 | 72.5 | 17.8 | 17.4 | 2.2 |
| Stage 4 | 36.3 | 13.0 | 28.5 | 11.3 |
| Stage 5 | 25.9 | 5.5 | 21.5 | 16.3 |
| Stage 6 | 30.3 | 2.2 | 8.6 | 10.3 |
| Stage 7 | 20.5 | 2.5 | 9.4 | 7.9 |
| MOC | 11.9 | 1.9 | 7.6 | 6.6 |

Table 8 shows a decrease in variability, as demonstrated by lower standard deviation, from the 60-hour rest period compared to the 20-hour rest period for both the 20 mg and 40 mg capsules of Blend 3.

### Blend 4 (2 kg Scale)

A 0.6 wt % magnesium stearate excipient preblend, i.e., lactose:MgSt, was prepared by combining lactose, 6% fines (3976.6 g) with magnesium stearate (23.2 g). A 6.5 wt % magnesium stearate API preblend, i.e., API:MgSt, was prepared by combining Compound I (243.3 g) with magnesium stearate (16.9 g). In a subsequent step, a U5 Comil® with a 457 mcm sieve was used to process 1792.3 g of the lactose:MgSt preblend and 175.9 g of the API:MgSt preblend at 1000 rpm into a 5 L TRV bowl to produce a mixture. The resulting mixture was then homogenized in a TRV blender at 1385 rpm for 7 minutes to afford a 10 wt % API bulk powder formulation (Blend 3). The Blend 4 bulk powder was then transferred to a clean, sealed GMP stainless-steel container (160 mm×160 mm) which followed with a resting period of 60 hours prior to capsule filling. Size 3 HPMC capsules were hand-filled with 20 mg of the Blend 4 bulk powder for a 2.0 mg target dose and 40 mg of the Blend 3 bulk powder for a 4.0 mg target dose.

The resulting composition of the 2.0 mg and 4.0 mg product is shown in Table 9.

TABLE 9

| | Composition |
|---|---|
| Component | (wt %) |
| Compound I | 10.3 |
| Magnesium Stearate | 1.2 |
| Lactose | 88.5 |

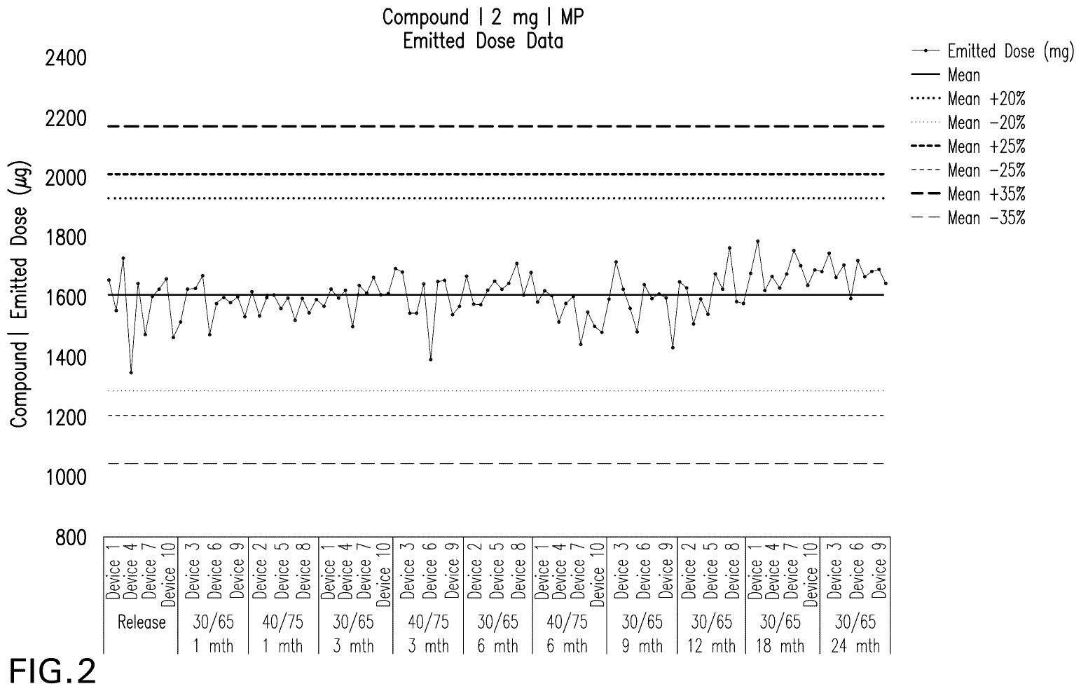

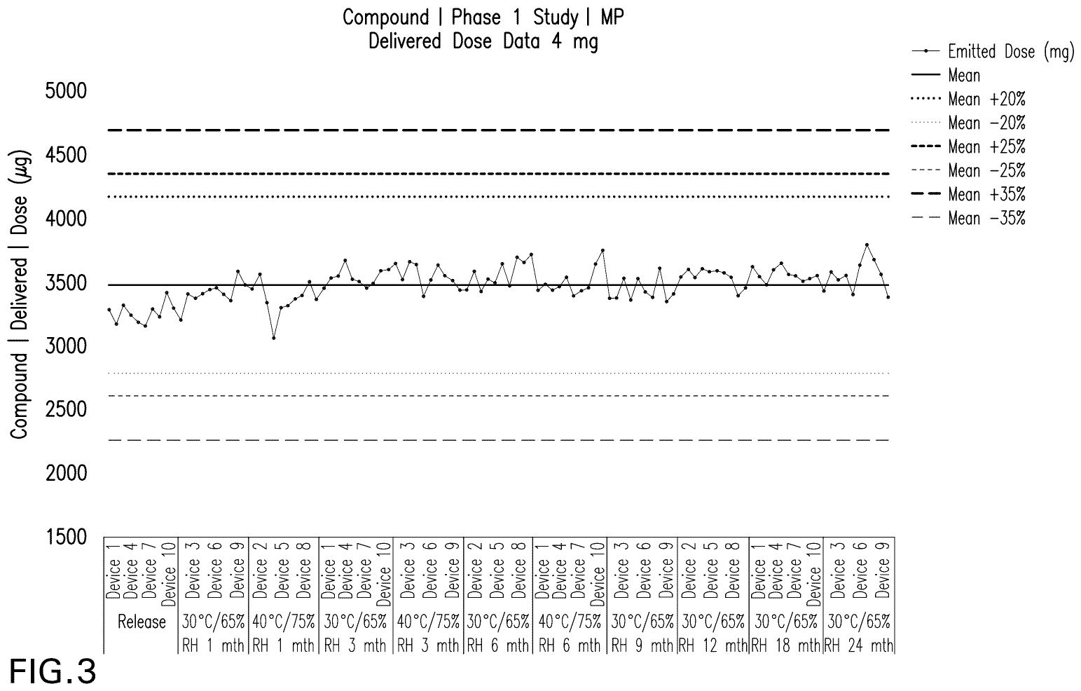

Content uniformity and drug content are provided in Table 10. Fine particle mass, fine particle fraction, and mass median aerodynamic diameter as determined for 2 mg and 4 mg capsules are provided in Table 11 and Table 12, respectively. A summary of NGI FPF data for marketed DPI products is provided in Table 13 for comparison. A summary of MMAD values for marketed DPI products is provided in Table 14 for comparison. Delivered dose data for 2 and 4 mg formulations of Compound I are provided in FIG. 2 and FIG. 3, respectively.

TABLE 10

| | Acceptance | |
|---|---|---|
| Test | Criteria | Result |
| Content Uniformity | ≤5.0% RSD | 0.7 |
| Drug Content | 8.5-11.5 wt % | 8.9 |

TABLE 11

| 2.0 mg | | 1 month | | 3 months | | 6 months | | 9 months | 12 months | 18 months | 24 months |
|---|---|---|---|---|---|---|---|---|---|---|---|
| Compound I | Release | 30/65 | 40/75 | 30/65 | 40/75 | 30/65 | 40/75 | 30/65 | 30/65 | 30/65 | 30/65 |
| Fine particle | 1006.7 | 1143.6 | 1046.6 | 1096.5 | 1090.9 | 1074.3 | 1069.6 | 1075.9 | 1098.4 | 1162.7 | 1083.1 |
| mass (≤5 μm) | | | | | | | | | | | |
| FPF % | 73.5 | 75.4 | 75.9 | 77.2 | 77.2 | 74.9 | 74.7 | 76.6 | 77.3 | 77.0 | 78.0 |
| (FPM/Impactor | | | | | | | | | | | |
| dose | | | | | | | | | | | |
| MMAD | 2.0 | 2.0 | 1.9 | 1.9 | 1.8 | 1.9 | 1.9 | 1.9 | 1.9 | 1.9 | 2.0 |

TABLE 12

| 4.0 mg | | 1 month | | 3 months | | 6 months | | 9 months | 12 months | | |
|---|---|---|---|---|---|---|---|---|---|---|---|
| Compound I | Release | 30/65 | 40/75 | 30/65 | 40/75 | 30/65 | 40/75 | 30/65 | 30/65 | 18 months | 24 months |
| Fine particle | 2157.0 | 2181.6 | 2130.4 | 2167.8 | 2059.2 | 2098.3 | 2090 | 2116.2 | 2182.2 | 2254.8 | 2201.8 |
| mass (≤5 μm) | | | | | | | | | | | |
| FPF % | 75.3 | 75.1 | 73.6 | 75.4 | 72.9 | 73.6 | 74 | 74 | 76.5 | 75.4 | 76.7 |
| (FPM/Impactor | | | | | | | | | | | |
| dose) | | | | | | | | | | | |
| MMAD | 1.9 | 1.8 | 1.9 | 1.8 | 1.9 | 1.9 | 1.8 | 1.8 | 1.9 | 1.9 | 1.9 |

The results from Table 11 (2.0 mg of Compound I) and Table 12 (4.0 mg of Compound I) show that formulations of Compound I demonstrate favorable drug delivery efficiency as evidenced by an FPF of approximately 73-78%. Table 11 and Table 12 also show that when formulations of Compound I are aerosolized, low MMAD values are achieved (1.8-2.0 μm). These data also suggest that the drug delivery efficiency is highly reproducible for formulations of Compound I stored at accelerated conditions for periods up to and including 24 months.

TABLE 13

| | | | | | | Multi-/ |
|---|---|---|---|---|---|---|
| | ICS | Manu- | % FPF at 4 kPa | | | single- |
| Inhaler | or BD | facturer | Defined as | | % FPF | unit dose |
| Flixotide | FLU | GSK | <5 | μm | 20-25 | Multi-unit |
| Diskus | | | | | | |
| Seretide | FLU | GSK | <5 | μm | 20-25 | Multi-unit |
| Diskus | | | | | | |
| Flixotide | FLU | GSK | <5 | μm | 25-30 | Multi-unit |
| Diskhaler | | | | | | |
| Rolenium | FLU | Elpen | <5 | μm | 15-20 | Single-unit |
| Elpenhaler | | | | | | |
| Pulmicort | BUD | AstraZeneca | <5 | μm | 30-35 | Multi-unit |
| Turbuhaler | | | | | | |
| Symbicort | BUD | AstraZeneca | <5 | μm | 45-50 | Multi-unit |
| Turbuhaler | | | | | | |
| Budelin | BUD | Meda | <5 | μm | 40-45 | Multi-unit |
| Novolizer | | | | | | |
| Budesonide | BUD | Sandoz | <5 | μm | 20-25 | Multi-unit |
| Easyhaler | | | | | | |
| Budesonide | BUD | Merck | <5 | μm | 10-15 | Multi-unit |
| Clickhaler | | Generics | | | | |
| Budesonide | BUD | Teva Pharma | <5 | μm | 25-30 | Single-unit |
| Cyclohaler | | | | | | |
| Budesonide | BUD | Ratiopharm | <5.1 | μm | 35-40 | Multi-unit |
| Jethaler | | | | | | |
| Foster | BDP | Chiesi | <5 | μm | 40-45 | Multi-unit |
| NEXThaler | | | | | | |
| Asmanex | MOM | Merck & Co | <5 | μm | 30-35 | Multi-unit |
| Twisthaler | | | | | | |
| Serevent | SAL | GSK | <5 | μm | 20-25 | Multi-unit |
| Diskus | | | | | | |
| Seretide | SAL | GSK | <5 | μm | 20-25 | Multi-unit |
| Diskus | | | | | | |
| Salbutamol | SAL | Teva | <5 | μm | 25-30 | Single-unit |
| Cyclohaler | | | | | | |
| Oxis | FOR | AstraZeneca | <5.8 | μm | 35-40 | Multi-unit |
| Turbuhaler | | | | | | |
| Symbicort | FOR | AstraZeneca | <5 | μm | 40-45 | Multi-unit |
| Turbuhaler | | | | | | |
| Formatris | FOR | Meda | <5 | μm | 40-45 | Multi-unit |
| Novolizer | | | | | | |
| Rolenium | FOR | Elpen | <5 | μm | 15-20 | Single-unit |
| Elpenhaler | | | | | | |
| Foster | FOR | Chiesi | <5 | μm | 35-40 | Multi-unit |
| NEXThaler | | | | | | |
| Foradil | FOR | Merck & Co. | <5.8 | μm | 25-30 | Single-unit |
| Aerolizer | | | | | | |
| Onbrez | IND | Novartis | <5 | μm | 35-40 | Single-unit |
| Breezhaler | | | | | | |
| Seebri | GB | Novartis | <5 | μm | 32-52 | Single-unit |
| Breezhaler | | | | | | |
| Eklira | ACC | Almirall | <5 | μm | 35-40 | Multi-unit |
| Genuair | | | | | | |
| Spiriva | TIO | Boehringer I | <5 | μm | 15-20 | Single-unit |
| HandiHaler | | | | | | |

BUD, budesonide; BDP, beclomethasone (dipropionate); FLU, flutisone (propionate); FPF, fine particle fraction; ICS, inhaled corticosteroid; MOM, mometasone (furoate); ACC, aclidinium (bromide); BD, bronchodilator; FOR, formoterol (fumarate dihydrate); GB, glycopyrronium (bromide); IND: indacaterol (maleate); SAL, salmeterol (xinafoate); TIO, tiotropium (bromide).

Also, the FPF data generated from formulations comprising Compound I (2.0 mg, Table 11 and 4.0 mg, Table 12) are significantly higher than that reported for a range of marketed dry powder inhaler products (Table 13) (Demoly et al., Respir. Med. 2014, 108, 1195).

TABLE 14

| DPI | MMAD (μm) |
|---|---|
| 2 mg formulation of Compound I | 1.8-2.0 |
| 4 mg formulation of Compound I | 1.8-1.9 |
| fluticasone propionate | 6-7 |
| budesonide | 2-3 |

Further to the above, the MMAD values generated from formulations comprising Compound I (2 mg, Table 11 and 4 mg, Table 12) are lower than that reported for marketed dry powder inhaler products (Table 14) (Derendorf et al., Eur. Respir. J. 2006, 28, 1042).

In addition, both the 2 mg and 4 mg formulations comprising Compound I demonstrate high drug delivery efficiency as shown by the delivered dose data in FIG. 2 and FIG. 3, respectively. As shown in FIG. 2, the mean delivered dose for the 2 mg formulation comprising Compound I is approximately 1.6 mg, or 80%. As shown in FIG. 3, the mean delivered dose for the 4 mg formulation comprising Compound I is approximately 3.47 mg, or 87%. All individual results are well within +/−20% of the mean delivered dose for both the 2 mg and 4 mg formulations. Further, the maximum and minimum deviation from the mean delivered dose are within 10% and 16%, respectively for the 2 mg formulation, and 9% and 12%, respectively for the 4 mg formulation. Thus, formulations comprising Compound I demonstrate exceptional drug delivery efficiency and reproducibility, which is independent of storage time and condition.

### Example 4: Animal Studies of Formulations of Compound I

Lactose/MgSt-based formulations of Compound I were evaluated during a 13-week inhalation toxicology study using a rat model.

The results from the toxicology study are presented in Table 15 below. No treatment-related effects on clinical signs, bodyweight, food consumption, ophthalmoscopy, blood chemistry, urinalysis or macroscopic pathology were observed. The NOAEL was assigned to the highest dose study (11.9 mg/kg/day).

TABLE 15

| | | | # animals | | | | | | AUC₀₋₂₄ | |
|---|---|---|---|---|---|---|---|---|---|---|
| | Target Delivered | | | | | | TK | | (h*ng/mL) | |
| | Compound I dose | MMAD | Main study | | Recovery | | satellite | | Day | Week |
| Group | (mg/kg/day) | (μm) | (M/F) | | (M/F) | | (M/F) | | 1 | 13 |
| 1 (control) | 0 | — | 10 | 10 | 5 | 5 | 3 | 3 | — | — |
| 2 (low dose) | 1.3 | 1.9 | 10 | 10 | 0 | 0 | 6 | 6 | 197 | 339 |
| 3 (mid dose) | 4.0 | 2.0 | 10 | 10 | 0 | 0 | 6 | 6 | 738 | 850 |
| 4 (high dose) | 12.0 | 2.1 | 10 | 10 | 5 | 5 | 6 | 6 | 1950 | 2060 |

Lactose/MgSt-based formulations of Compound I were evaluated during a 13-week inhalation toxicology study in an NHP model, and the corresponding results are presented in Table 16 below. No treatment-related effects on clinical signs, bodyweight, food consumption, ophthalmoscopy, blood chemistry, urinalysis or macroscopic pathology were observed. The NOAEL was assigned to the highest dose study (7.9 mg/kg/day).

TABLE 16

| | Target Delivered | | # animals | | | | AUC₀₋₂₄ | |
|---|---|---|---|---|---|---|---|---|
| | Compound I dose | MMAD | Main study | | Recovery | | (h*ng/mL) | |
| Group | (mg/kg/day) | (μm) | (M/F) | | (M/F) | | Day 1 | Week 13 |
| 1 (control) | 0 | | 3 | 3 | 2 | 2 | 0 | tbc* |
| 2 (low dose) | 0.9 | 1.9 | 3 | 3 | 0 | 0 | 203 | 167 |
| 3 (mid dose) | 2.7 | 2.3 | 3 | 3 | 0 | 0 | 408 | 361 |
| 4 (high dose) | 8.0 | 2.7 | 3 | 3 | 2 | 2 | 903 | 1160 |

Results from the margin of safety study of lactose/MgSt-based formulations of Compound I are shown below in Table 17. The results suggest that there are significant systemic and lung margins of safety established versus the highest daily (4 mg BID) pharmacologically active dose.

TABLE 17

| | Group mean AUC₀₋₂₄ MoS | | | |
|---|---|---|---|---|
| | (Phase 1 clinical study versus | | Lung dose of | |
| Animal | inhalation tox study NOAELs) | | Compound I (mg/g)² MoS | |
| study | 13-wk rat | 13-wk NHP | 13-wk rat | 13-wk NHP |
| repeat dose | NOAEL | NOAEL | NOAEL | NOAEL |
| 4.0 mg BID | 5.6 | 3.2 | 25 | 50 |

²Adjusted for respirability assuming 10%, 25% and 100% lung deposition in rat, NHP and human.

### Example 5: Phase 1 Clinical Trials of Formulations of Compound I

A status summary of the phase 1 clinical trial recruitment is shown below in Table 18. Capsules filled with blend 2a were used to achieve a 0.6 mg target dose of Compound I (0.2 mg capsules×3). Capsules filled with blend 4 were used to achieve 2.0 mg, 4.0 mg, 6.0 mg, 8.0 mg, and 12.0 mg target doses of Compound I (2.0 mg capsule×1; 4.0 mg capsule×1; 2.0 mg capsules×3; 4.0 mg capsules×2; and 4.0 mg capsules×3, respectively). Twice daily dosing (BID) was achieved by repeating the preceding regime. For example, an 8.0 mg BID dosing regime is achieved by administering two doses of 4.0 mg capsules×2.

TABLE 18

| | | | # subjects | | |
|---|---|---|---|---|---|
| | | | (active:placebo)/ | Dose of | |
| Part | Cohort | Subject ID | cohort | Compound I | Status |
| 1 | 1 | Healthy | 6:2 | 0.2 mg/placebo | Completed |
| | 2 | volunteers | | 0.6 mg/placebo | |
| | 3 | | | 2.0 mg/placebo | |
| | 4 | | | 4.0 mg/placebo | |
| | 5 | | | 6.0 mg/placebo | |
| | 6 | | | 12.0 mg/placebo | |
| 2 | 1 | ICS-naïve stable | | 0.6 QD mg/placebo | Completed |
| | 2 | mild asthmatics | | 2.0 mg QD/placebo | |
| | 3 | | | 4.0 mg BID/placebo | |
| | 4 | | | 8.0 mg BID/placebo | |
| 3 | 1 | Moderate to severe | 17:6 | 4.0 mg BID/placebo | Completed |
| | | asthma | | | |

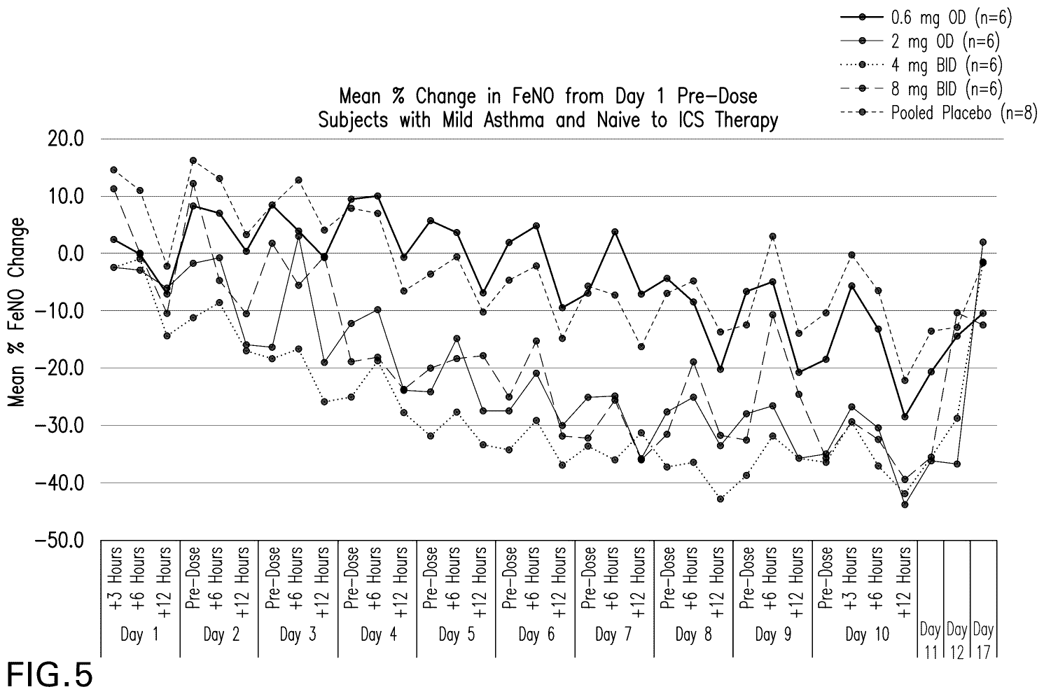

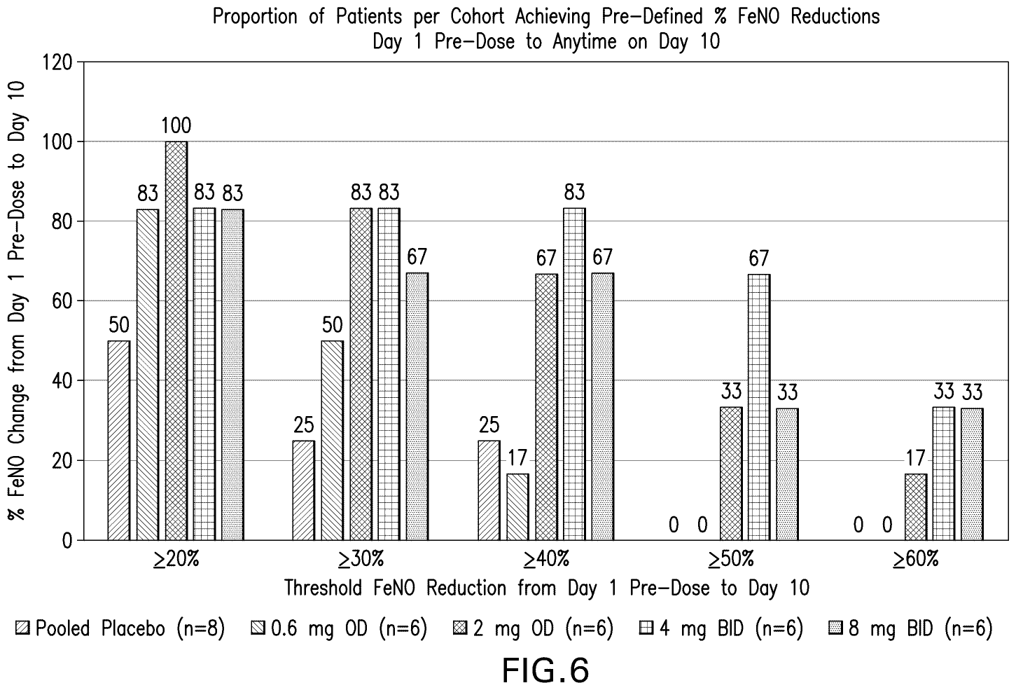

Particular FeNO reduction data from FIG. 5 is highlighted below in Table 19. For comparison, it is established that FeNO reductions greater than 20% are clinically significant (Am. J. Respir. Crit. Care Med. 2011, 184, 602). The results suggest that formulations comprising Compound I at doses greater than or equal to 2.0 mg QD delivered via a single capsule provide clinically relevant FeNO reduction.

TABLE 19

Mean placebo-corrected
% FeNO reduction from
day 1 pre-dose in ICS
naïve mild asthmatics

| Dose of | Day 10 + | Day 10 + |
|---|---|---|
| Compound I | 3 hrs | 6 hrs |
| 0.6 mg QD (n = 6) | −5.4 | −6.7 |
| 2.0 mg QD (n = 6) | −26.6 | −24.0 |
| 4.0 mg BID (n = 6) | −29.1 | −30.6 |
| 8.0 mg BID (n = 6) | −29.1 | −25.9 |

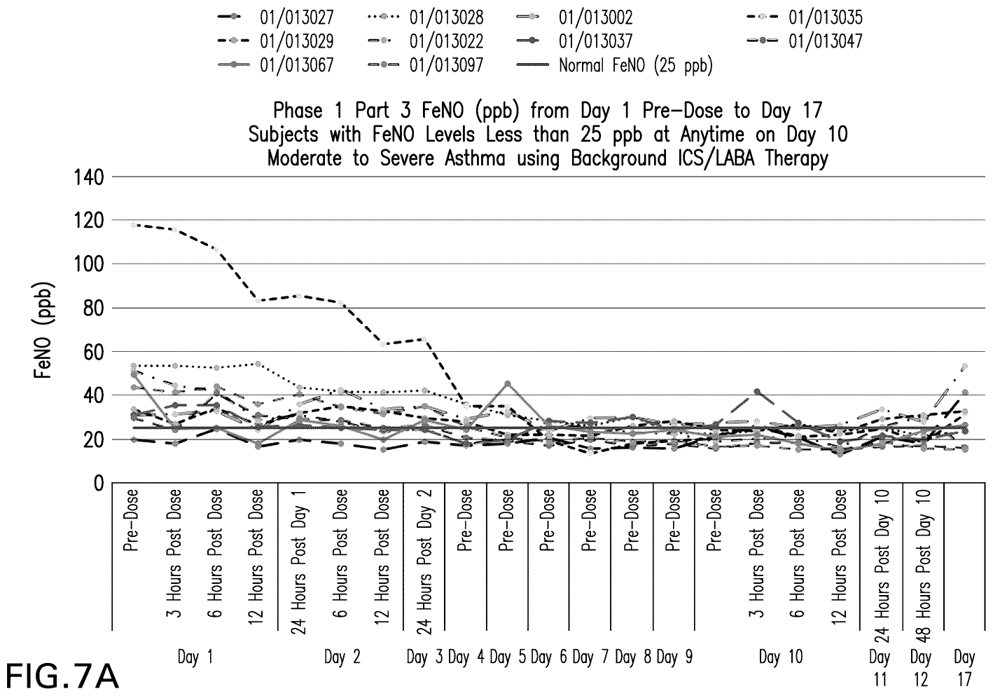

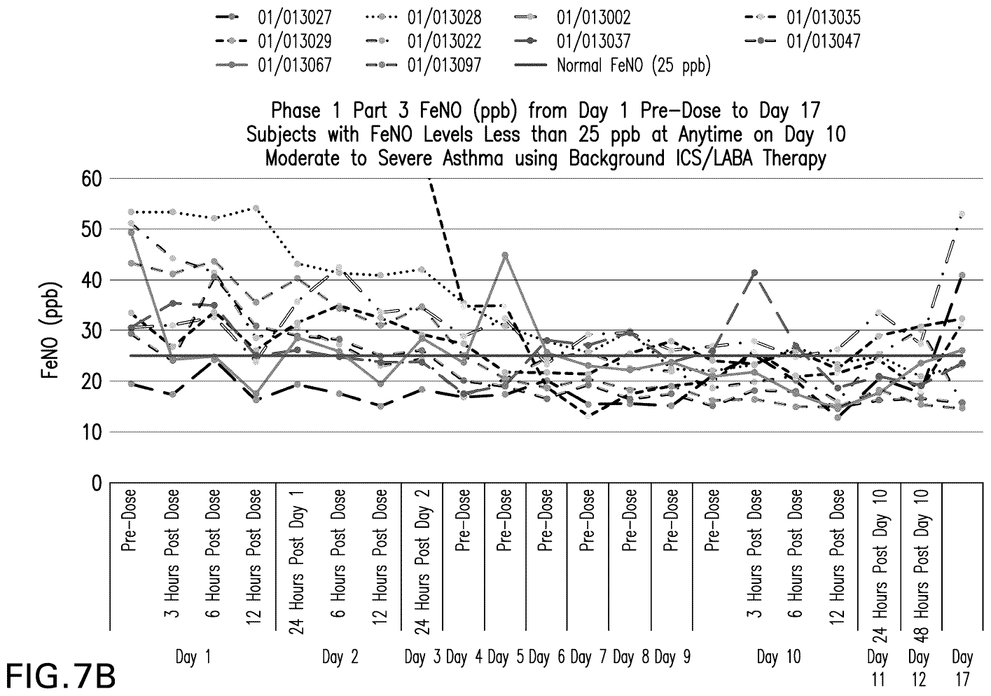

The proportion of subjects with FeNO level <25 ppb on the final day of dosing (Day 10) from FIG. 7 is highlighted below in Table 20. These results further suggest that formulations comprising Compound I at doses greater than or equal to 2.0 mg QD delivered via a single capsule provide clinically relevant FeNO reduction.

TABLE 20

| | % subjects reporting |
|---|---|
| Dose of | Day 10 FeNO value |
| Compound I | <25 ppb |
| Pooled placebo (n = 8) | 13 |
| 0.6 mg QD (n = 6) | 16 |
| 2.0 mg QD (n = 6) | 16 |
| 4.0 mg BID (n = 6) | 67 |
| 8.0 mg BID (n = 6) | 50 |

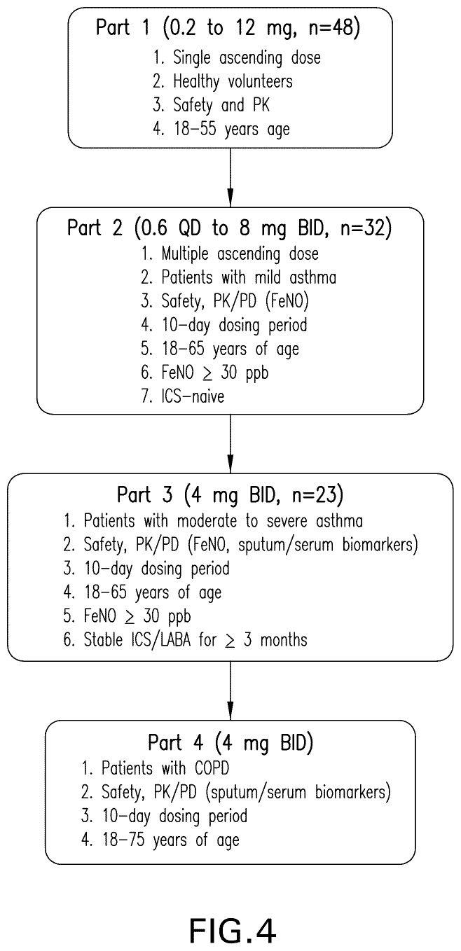

The adverse events summary from the Compound I phase 1 clinical trial (FIG. 4) is provided in Table 21. No severe or serious adverse events were observed, only adverse events of mild/moderate severity were observed and typically with an “unlikely relationship.” No clinically relevant changes per investigator in hematology, biochemistry or urinalysis parameters including those influenced by JAK inhibition (e.g., WBC, platelets, reticulocytes, APTT, lipids, absolute neutrophil counts) were observed. No treatment-related clinically relevant changes in cardiovascular endpoints (i.e., ECG and vital signs) were observed.

TABLE 21

| | Part 1 Cohorts (n = 6 active, n = 2 placebo per cohort)¹ | | | | | | |
|---|---|---|---|---|---|---|---|
| | 0.2 mg | 0.6 mg | 2.0 mg | 4.0 mg | 6.0 mg | 12.0 mg | Placebo |
| AE summary | (n = 6) | (n = 7) | (n = 6) | (n = 6) | (n = 6) | (n = 6) | (n = 12) |
| Subjects with | 0 | 2 (29%) | 2 (33%) | 0 | 0 | 0 | 4 (33%) |
| TEAE n, (%) | | | | | | | |
| Severe/serious | 0 | 0 | 0 | 0 | 0 | 0 | 0 |
| TEAE, n (%) | | | | | | | |
| TEAE leading to | 0 | 1 | 0 | 0 | 0 | 0 | 0 |
| discontinuation | | | | | | | |

The FeNO reduction data compared to the margin of safety is presented below in Table 22. The data suggests that a dose of 4.0 mg BID represents the best chance of achieving clinical efficiency in phase 2 clinical trials.

TABLE 22

| | Mean placebo-corrected | | | MoS versus inhalation toxicology study NOAELs | | | |
|---|---|---|---|---|---|---|---|
| | % FeNO reduction from | | | Plasma (group | | Lung exposure | |
| | Day 1 pre-dose | | # | mean AUC₀₋₂₄) | | (mg/g lung) | |
| | Day 10 + | Day 10 + | capsules | 13-week | 13-week | 13-week | 13-week |
| Dose | 3 hours | 6 hours | per dose | rat study | NHP study | rat study | NHP study |
| 4.0 mg BID | 29.1 | 30.6 | 1 | 85.6 | 3.2 | 3925 | 50 |

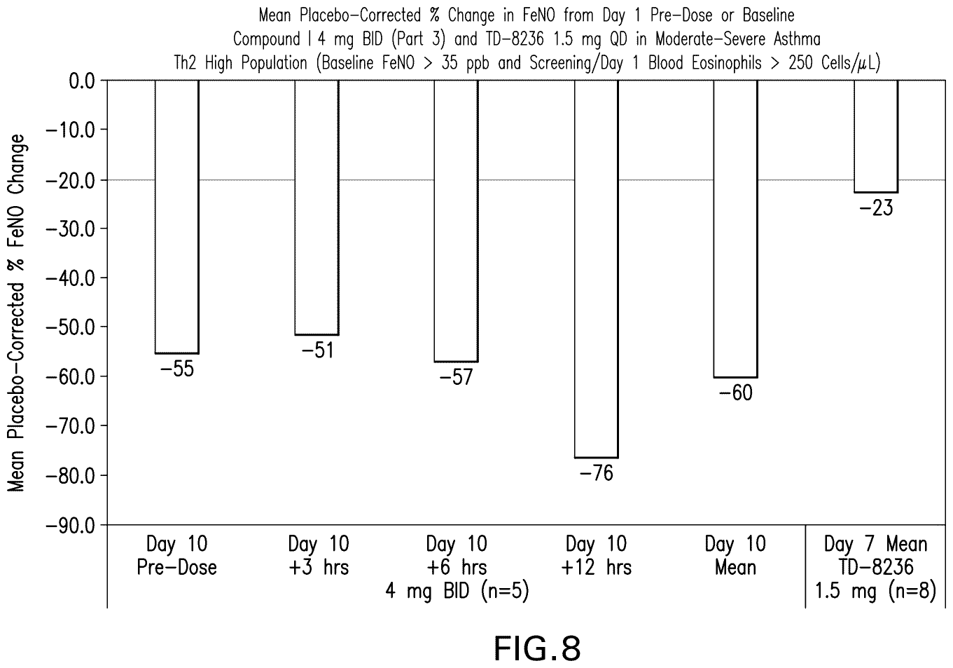

A comparison of the FeNO reduction data obtained with formulations of Compound I obtained under similar conditions compared to formulations comprising JAK1 inhibitors GDC-0214 and GDC-4379 are presented below in Table 23. A similar comparison between formulations comprising Compound I and formulations comprising TD-8236 (pan JAK inhibitor) is presented in FIG. 8, with the corresponding data highlighted in Table 24. It is noted that that the patients administered formulations comprising Compound I, GDC-0214, and GDC-4379 are characterized with mild asthma and are naïve to ICS therapy, and the patients administered TD-8236 are characterized with moderate to severe asthma.

The data suggests that clinically relevant FeNO reduction can be achieved using formulations comprising Compound I at lower API doses and with fewer capsules compared to formulations comprising GDC-0214 and formulations comprising GDC-4379. The data also shows that formulations comprising Compound I demonstrate significantly greater FeNO reductions at a dose administered via a single capsule compared to formulations comprising TD-8236 in patients with moderate to severe asthma.

TABLE 23

| | Formulations | | | Formulations | | | | Formulations | | | |
|---|---|---|---|---|---|---|---|---|---|---|---|
| | Comprising Compound I | | | Comprising GDC-0214 | | | | Comprising GDC-4379 | | | |
| | 0.6 mg | 2.0 mg | 4.0 mg | 1.0 mg | 4.0 mg | 15.0 mg | 15.0 mg | 10.0 mg | 30.0 mg | 40.0 mg | 80.0 mg |
| Dose regime | QD | QD | BID | QD | QD | QD | BID | QD | QD | BID | QD |
| Mean placebo- | −7% | −24% | −26% | 0.4%* | −6.5% | −23% | −42% | −5.7% | −26% | −55% | −52% |
| corrected FeNO | | | | | | | | | | | |
| reduction | | | | | | | | | | | |
| # of capsules | 3 | 1 | 1 | 1 | 4 | 15 | 30 | 1 | 3 | 4 | 8 |
| per dose | | | | | | | | | | | |

*an increase in FeNO was observed

TABLE 24

| | | Mean Placebo-Corrected % Change |
|---|---|---|
| | | from Day 1 Pre-Dose or from |
| Dose | Time | Patient's Baseline¹ |
| Compound I | Day 10 pre-dose | −44 |
| 4 mg BID (n = 5) | Day 10 + 3 hrs | −37 |
| | Day 10 + 6 hrs | −43 |
| | Day 10 + 12 hrs | −47 |
| | Day 10 mean | −43 |
| TD-8236 1.5 mg (n = 8) | Day 7 mean | −23 |

¹subjects with Baseline FeNO >35 ppb and Screening/Day 1 blood eosinophil levels >250 cells/μL

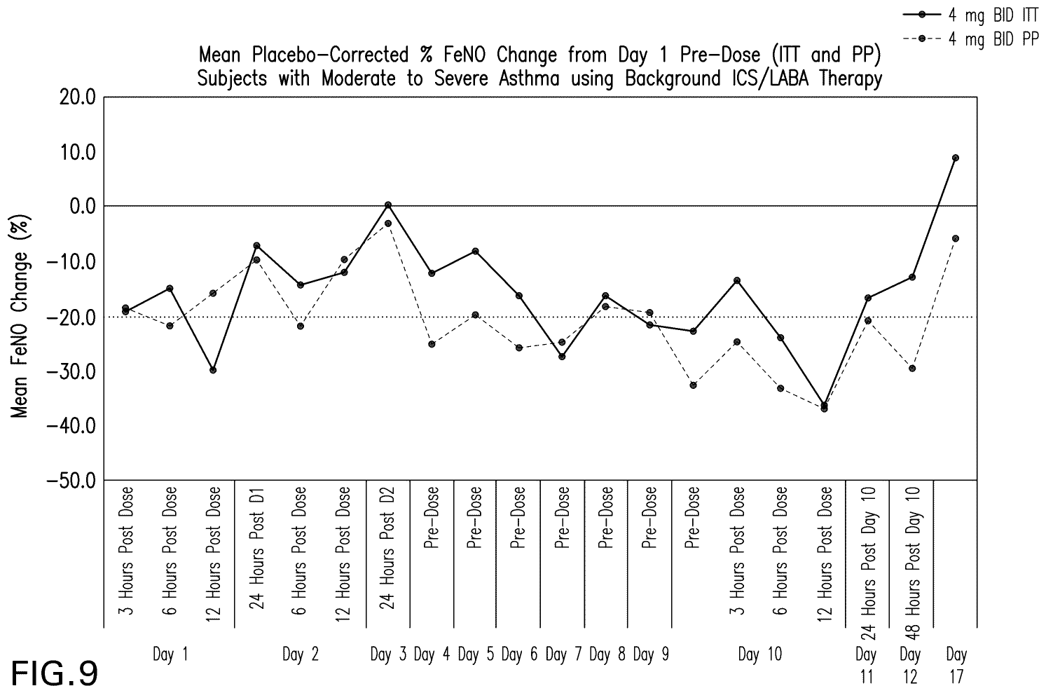

Particular FeNO reduction data from FIG. 9 is highlighted below in Table 25. The results suggest that clinically relevant FeNO reductions are achieved in patients with moderate to severe asthma who use background ICS/LABA therapy post administration of a 4 mg BID regimen of formulations comprising Compound I. It is emphasized that each dose was delivered via a single capsule over the 10-day period. More specifically, subjects with Day 1 pre-dose FeNO levels between 33 and 280 ppb reported reductions greater than 20% by Day 10 and subjects with Day 1 pre-dose FeNO levels ranging from 33 to 118 ppb recorded levels less than 25 ppb by Day 10.

TABLE 25

| | |
|---|---|
| | Mean placebo-corrected |
| | % FeNO reduction from |
| Time Post Dose | Day 1 pre-dose |

| of Compound I | ITT (n = 17) | PP¹ (n = 15) |
|---|---|---|
| Day 10 pre-dose | −22.9 | −32.7 |
| Day 10 + 3 hours | −13.7 | −24.8 |
| Day 10 + 6 hours | −24.0 | −33.3 |
| Day 10 + 12 hours | −36.3 | −37.0 |
| Day 10 mean | −24.2 | −31.9 |

¹per protocol excludes subjects with Day 1 pre-dose FeNO level <30 ppb

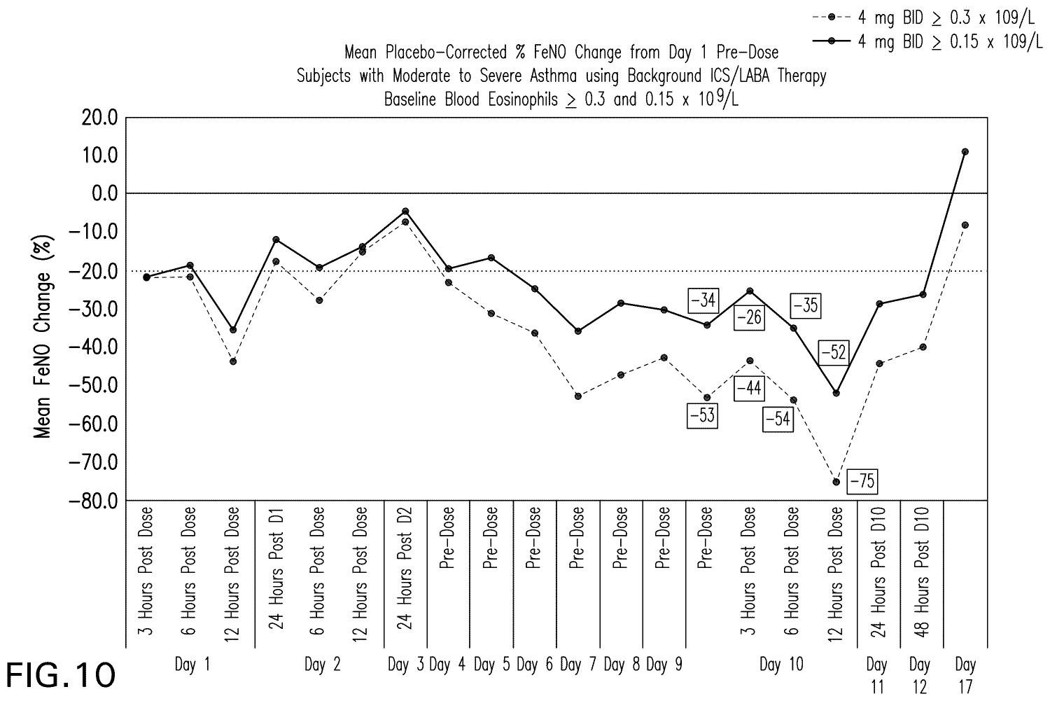

Particular FeNO reduction data from FIG. 10 is highlighted below in Table 26 where greater reductions in FeNO are achieved in subjects with elevated blood eosinophil levels at baseline.

TABLE 26

Blood eosinophil cut-off

| Time Post Dose of | ≥0.3 × 10⁹/L | ≥0.15 × 10⁹/L |
|---|---|---|
| Compound I | (n = 11) | (n = 16) |
| Day 10 pre-dose | −53 | −34 |
| Day 10 + 3 hours | −44 | −26 |
| Day 10 + 6 hours | −54 | −35 |
| Day 10 + 12 hours | −75 | −52 |
| Day 10 mean | −57 | −37 |

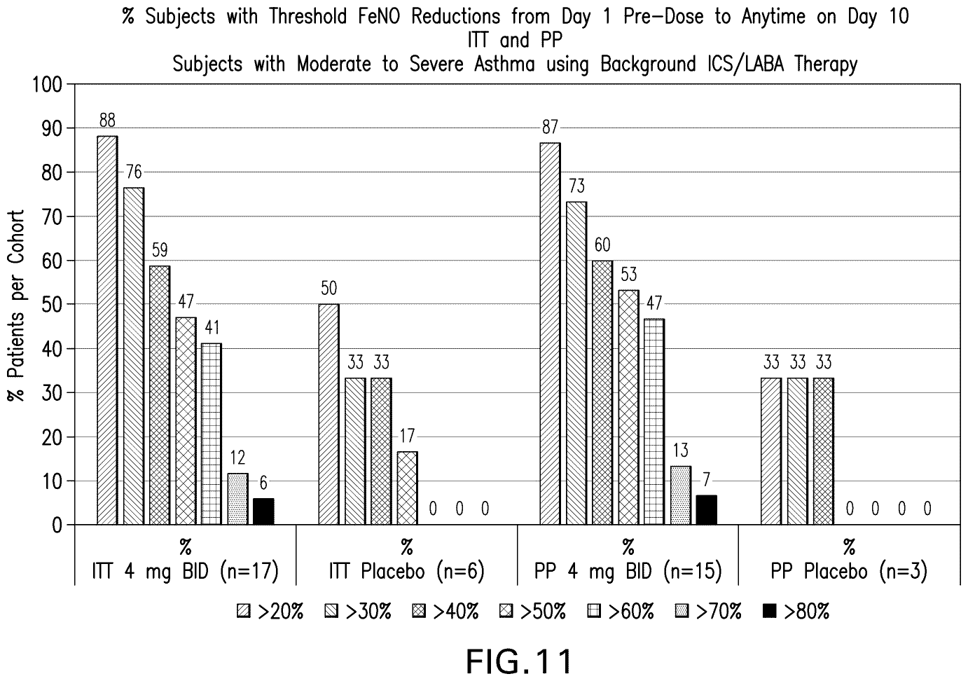

Particular FeNO reduction data from FIG. 11 is highlighted below in Table 27. These results further suggest that formulations comprising Compound I at doses of 4.0 mg BID provide clinically relevant FeNO reduction in patients with moderate to severe asthma using background ICS/LABA therapy.

TABLE 27

| | |
|---|---|
| | % subjects |
| | achieving FeNO level |
| Time Post Dose of | <25 ppb on Day 10 |

| Compound I | ITT | PP¹ |
|---|---|---|
| Active | 59 | 52 |
| Placebo | 33 | 0 |

¹excludes subjects with Day 1 pre-dose FeNO level <30 ppb

Day 10 pharmacokinetic parameters for Parts 2 and 3 of Phase I are summarized below in Table 28. Part 2 involved the administration of 4 different dose regimens, to subjects with mild asthma naïve to ICS therapy, over a 10-day period. Each separate cohort comprised 6 subjects receiving active treatment with 2 receiving matching placebo. Blood samples were collected to enable the measurement of Compound I concentrations in plasma. The resultant data was used to determine the PK parameters at Day 10 representing the Day on which the final dose was received. Dose regimens from 2 mg QD to 8 mg BID were considered to be pharmacologically active as evidenced by clinically relevant mean placebo-corrected changes in FeNO from Day 1 pre-dose to Day 10. The 0.6 mg QD was identified as a minimally active dose where mean placebo-corrected FeNO changes were below the 20% threshold used to determine clinical relevance.

Part 3 involved the administration of a single dose regimen (4 mg BID), to subjects (17 active:6 placebo) with moderate to severe asthma treated with background ISC/LABA therapy, over a 10-day period. Blood samples were collected to enable the measurement of KN-002 concentrations in plasma. The resultant data was used to determine the pharmacokinetic parameters at Day 10 representing the Day on which the final dose was received.

TABLE 28

| | Study Part | | | | |
|---|---|---|---|---|---|
| | | | | | 3 (moderate to |
| | 2 (mild asthma) | | | | severe asthma) |
| | 0.6 mg | 2 mg | 4 mg | 8 mg | 4 mg |
| PK parameter at Day 10 | QD | QD | BID | BID | BID |
| AUC_{0-∞} | | | | | |
| (pg · h/ml) | | | | | |
| Mean | 28,042 | 89,167 | 358,678 | 746,660 | 439,258 |
| Geometric mean | 24,292 | 83,866 | 281,796 | 663,941 | 394,517 |
| Max | 52,842 | 145,552 | 587,424 | 1,369,630 | 887,512 |
| Min | 8,783 | 50,025 | 44,654 | 358,998 | 134,831 |
| AUC_{0-last non-zero} | | | | | |
| (pg · h/ml) | | | | | |
| Mean | 26,731 | 87,379 | 354,781 | 744,675 | 437,767 |
| Geometric mean | 23,385 | 82,180 | 277,609 | 661,886 | 392,724 |
| Max | 47,928 | 143,585 | 586,513 | 1,369,314 | 886,864 |
| Min | 8,579 | 48,931 | 43,356 | 358,137 | 133,118 |
| AUC_{over dosing interval} | | | | | |
| (pg · h/ml) | | | | | |
| Mean | 22,518 | 76,444 | 236,794 | 458,169 | 278,313 |
| Geometric mean | 20,283 | 71,866 | 193,563 | 417,623 | 257,114 |
| Max | 36,466 | 125,837 | 373,995 | 830,085 | 501,523 |
| Min | 8,579 | 42,693 | 39,111 | 260,305 | 105,203 |
| AUC_{0-12 hours} | | | | | |
| (pg · h/ml) | | | | | |
| Mean | 16,093 | 55,247 | 236,794 | 458,169 | Not calculated |
| Geometric mean | 14,866 | 51,927 | 193,563 | 417,623 | |
| Max | 23,507 | 88,203 | 373,995 | 830,085 | |
| Min | 7,596 | 30,807 | 39,111 | 260,305 | |
| AUC_{0-24 hours} | | | | | |
| (pg · h/ml) | | | | | |
| Mean | 22,518 | 76,444 | 299,419 | 611,627 | 365,148 |
| Geometric mean | 20,283 | 71,866 | 241,352 | 544,711 | 333,807 |
| Max | 36,466 | 125,837 | 473,634 | 1,140,247 | 689,494 |
| Min | 8,579 | 42,693 | 43,350 | 309,050 | 123,642 |
| C_{trough} | | | | | |
| (pg/ml) | | | | | |
| Mean | 362 | 970 | 10,001 | 21,835 | 11,153 |
| Geometric mean | 234 | 929 | 6,983 | 17,666 | 9,806 |
| Max | 913 | 1,370 | 15,600 | 40,600 | 23,000 |
| Min | 34 | 536 | 563 | 6,090 | 2,490 |
| Cₘₐₓ | | | | | |
| (pg/ml) | | | | | |
| Mean | 1,965 | 6,457 | 31,458 | 60,433 | 37,547 |
| Geometric mean | 1,860 | 6,190 | 26,800 | 57,709 | 35,028 |
| Max | 2,750 | 9,630 | 47,800 | 91,500 | 63,800 |
| Min | 1,020 | 4,160 | 7,750 | 45,400 | 15,900 |

Day 10 sputum and serum inflammatory biomarkers are shown below in Table 29. Part 3 included an evaluation of sputum samples obtained by sputum induction in accordance with European Respiratory Society guidelines (Paggiaro, et al., Eur. Respir. J. 2002, 20: Suppl. 37, 3 s). Subjects were randomized into sputum-producer and non-sputum producer strata with a target minimum of approximately 50% of subjects able to provide an adequate sputum sample at screening. Subjects in the sputum producer stratum had a second sputum induction performed following 10 days of treatment with 4 mg Compound I BID or placebo. Differential cell counts (DCC) were performed on samples judged adequate per standard procedure and patients with evaluable baseline (screening) and Day 10 (or Day 11 if Day 10 was not adequate) post-treatment samples were considered for analyses that included individual and mean absolute and percentage differential eosinophil counts.

In the sputum producer stratum, 6 Compound I (active) subjects and 2 placebo subjects were identified as having adequate baseline and post-treatment sputum samples and the resultant data was used to determine change from baseline, mean change from baseline, and mean placebo-corrected change from baseline (Compound I vs placebo). As reported below, improvements (decreases) from baseline in both individual and mean absolute and percentage differential eosinophil counts were observed in all active-treated subjects while no improvements were observed for either parameter in placebo subjects. Placebo-corrected values for mean change from baseline showed corresponding improvements in absolute and percentage differential eosinophil counts. At baseline, all subjects manifested a percent differential eosinophil count >3%, indicative of active eosinophilic inflammation. By Day 10, 66% ( 4/6) of active subjects had improved below 3% eosinophils, with 50% ( 3/6) below a more stringent threshold of 1.9% (Reddel et al., Am. J. Respir. Crit. Care Med. 2009, 180, 59). No placebo subjects showed improvement. These findings confirm pharmacodynamic engagement of Compound I at the site of activity (airways) and demonstrate clinically meaningful effect on eosinophilic inflammation after 10 days of treatment.

Whole blood hematology samples were collected prior to dosing in all patients at Day 1 (baseline) and on Day 10. Parameters including whole blood absolute and percent differential eosinophil count were determined and the resultant data was used to determine change from baseline, mean change from baseline, and mean placebo-corrected change from baseline (Compound I vs placebo). As reported below, improvements (decreases) from baseline were observed in the 83% of active subjects for (⅚) with small decreases observed in both placebo subjects. Decreases (improvements) in mean absolute and percentage differential eosinophil counts were observed in active-treated subjects with placebo-corrected values for mean change from baseline similarly reflecting improvements in this marker of eosinophilic inflammation.

TABLE 29

| | | | | | | | | | | | Mean |
|---|---|---|---|---|---|---|---|---|---|---|---|
| | | | | | | | | | | | placebo- |
| | Active subject ID (n = 6) | | | | | | | Placebo subject ID (n = 2) | | | corrected |
| Time post dose | 13026 | 13027 | 13038 | 13073 | 13095 | 13097 | Mean | 13036 | 13053 | Mean | change |

Induced sputum % differential eosinophil count

| | | | | | | | | | | | |
|---|---|---|---|---|---|---|---|---|---|---|---|
| Screening level | 15.3 | 4.8 | 5.3 | 29.3 | 4.0 | 4.8 | 10.5 | 8.3 | 4.0 | 6.1 | |
| Day 10 level | 7.0 | 2.5 | 1.0 | 10.5 | 1.0 | 0.3 | 3.7 | 25.3 | 16.0 | 20.7 | |
| Change from Screening | −8.3 | −2.3 | −4.3 | −18.8 | −3.0 | −4.5 | −6.8 | 17.0 | 12.0 | 14.5 | −21.3 |
| % Change from Screening | −54 | −48 | −81 | −64 | −75 | −94 | −65 | +213 | +300 | 238 | |
| Reduced to normal level (<3%) | No | Yes | Yes | No | Yes | Yes | | No | No | | |

Induced sputum absolute differential eosinophil count (10⁶/g)

| | | | | | | | | | | | |
|---|---|---|---|---|---|---|---|---|---|---|---|
| Screening level | 0.23 | 0.12 | 0.09 | 0.44 | 0.08 | 0.17 | 0.19 | 0.36 | 0.04 | 0.2 | |
| Day 10 level | 0.07 | 0.03 | 0.01 | 0.12 | 0.01 | 0.001 | 0.04 | 0.50 | 0.05 | 0.30 | |
| Change from Screening | −0.16 | −0.09 | −0.08 | −0.33 | −0.07 | −0.17 | −0.15 | 0.14 | 0.004 | 0.07 | −0.22 |
| % Change from Screening | −70 | −75 | −89 | −75 | −88 | −100 | −79 | +39 | +10 | +25 | |

Serum absolute differential eosinophil count (cells 10⁹/L)

| | | | | | | | | | | | |
|---|---|---|---|---|---|---|---|---|---|---|---|
| Day 1 pre-dose level | 0.66 | 0.24 | 0.30 | 0.77 | 0.44 | 0.46 | 0.48 | 0.50 | 0.25 | 0.38 | |
| Day 10 level | 0.36 | 0.37 | 0.17 | 0.74 | 0.30 | 0.12 | 0.34 | 0.47 | 0.21 | 0.34 | |
| Change from Day 1 pre-dose | −0.30 | 0.13 | −0.13 | −0.03 | −0.14 | −0.34 | −0.14 | −0.03 | −0.04 | −0.04 | −0.11 |
| % Change from screening | −45 | +54 | −43 | −4 | −32 | −74 | −29 | −6 | −16 | −11 | |

Although the formulations of the disclosure have been described in some detail by way of illustration and example for purposes of clarity of understanding, one of ordinary skill in the art will appreciate that certain changes and modifications may be practiced within the scope of the appended claims. In addition, each reference, including all of the U.S. patents, U.S. patent application publications, U.S. patent applications, foreign patents, foreign patent applications and non-patent publications referred to in this specification are incorporated herein by reference, in their entirety, to the extent not inconsistent with the present description. Where a conflict exists between the instant application and a reference provided herein, the instant application shall dominate.

1. A pharmaceutical formulation comprising:
(a) (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile: 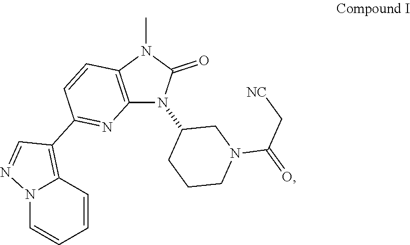
or a pharmaceutically acceptable salt thereof;
(b) magnesium stearate; and
(c) lactose.

2. The pharmaceutical formulation of claim 1, wherein the formulation comprises:
(a) 0.5-11.0 weight percent Compound I, or a pharmaceutically acceptable salt thereof;
(b) 0.5-2.0 weight percent magnesium stearate; and
(c) 87.0-99.0 weight percent lactose.

3. The pharmaceutical formulation of claim 1, wherein the formulation comprises:
(a) about 1.1 weight percent Compound I, or a pharmaceutically acceptable salt thereof;
(b) about 1.0 weight percent magnesium stearate; and
(c) about 97.9 weight percent lactose.

4. The pharmaceutical formulation of claim 1, wherein the formulation comprises:
(a) about 10.3 weight percent Compound I, or a pharmaceutically acceptable salt thereof;
(b) about 1.2 weight percent magnesium stearate; and
(c) about 88.5 weight percent lactose.

5-9. (canceled)

10. The pharmaceutical formulation of claim 1, wherein the content uniformity of the pharmaceutical formulation of Compound I has a percent relative standard deviation (RSD) less than or equal to about 5%.

11. The pharmaceutical formulation of claim 1, wherein the formulation is suitable for aerosolized delivery to a subject.

12. The pharmaceutical formulation of claim 11, wherein, when aerosolized, the mass median aerodynamic diameter (MMAD) is less than or equal to about 5.0 μm.

13. The pharmaceutical formulation of claim 11, wherein, when aerosolized, the fine particle fraction (FPF) of Compound I is greater than or equal to about 60%.

14. The pharmaceutical formulation of claim 11, wherein, when a capsule containing about 1.0-3.0 mg of Compound I is aerosolized, the fine particle mass (FPM) of Compound I is 900-1800 μg.

15. The pharmaceutical formulation of claim 11, wherein, when a capsule containing about 3.0-5.0 mg of Compound I is aerosolized, the FPM of Compound I is 1750-2350 μg.

16. The pharmaceutical formulation of claim 11, wherein, when aerosolized, the delivered dose of Compound I is at least about 80% of the target dose.

17. The pharmaceutical formulation of claim 11, wherein Compound I is a polymorph form having the following diffraction angles (2Theta) based on cupric Kα1:
at approximately 8.25°;
at approximately 13.25°;
at approximately 15.40°;
at approximately 17.65°; and
at approximately 25.39° (Form II).

18. A method of treating asthma, COPD, or an inflammatory condition associated with eosinophilic inflammation or non-eosinophilic inflammation in a subject in need thereof, wherein the method comprises administering to a subject the pharmaceutical formulation of claim 1.

19. (canceled)

20. The method of claim 18, wherein the inflammatory condition associated with eosinophilic inflammation or non-eosinophilic inflammation is eosinophilic asthma, nasal polyposis, rhinitis, pulmonary fibrotic disease, interstitial lung disease, or pulmonary hypertension.

21-22. (canceled)

23. The method of claim 18, wherein the subject's FeNO concentration is reduced to at least about 35 parts per billion (ppb).

24. The method of claim 23, wherein the subject's FeNO concentration is reduced to at least about 30 ppb.

25. The method of claim 23, wherein the subject's FeNO concentration is reduced to at least about 25 ppb.

26. The method of claim 23, wherein the subject's FeNO concentration is reduced to at least about 20 ppb.

27. The method of claim 23, wherein the subject's FeNO concentration is reduced relative to the subject's pre-administration FeNO concentration.

28. The method of claim 18, wherein the subject's FeNO concentration is reduced by at least about 20%.

29. The method of claim 28, wherein the subject's FeNO concentration is reduced by at least about 30%.

30. The method of claim 28, wherein the subject's FeNO concentration is reduced by at least about 40%.

31. The method of claim 28, wherein the subject's FeNO concentration is reduced by at least about 50%.

32. The method of claim 28, wherein the subject's FeNO concentration is reduced by at least about 60%.

33. The method of claim 28, wherein the subject's FeNO concentration is reduced by at least about 65%.

34. The method of claim 28, wherein the subject's FeNO concentration is reduced by at least about 80%.

35. The method of claim 28, wherein the subject's FeNO concentration is reduced relative to both the subject's pre-administration FeNO concentration and the FeNO concentration upon placebo treatment.

36. (canceled)

37. The method of claim 18, wherein the formulation is administered as a single capsule.

38. The method of claim 18, wherein the subject has moderate to severe asthma.

39. The method of claim 38, wherein the subject's moderate to severe asthma is characterized by a Th2-high phenotype.

40. A method for the preparation of a dry powder pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I), wherein the method comprises:
(a) combining (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile (Compound I), or a pharmaceutically acceptable salt thereof with magnesium stearate to form a first preblend;
(b) combining magnesium stearate and lactose to form a second preblend;
(c) combining the first preblend with the second preblend;
(d) co-milling the product formed in step (c); and
(e) blending the product formed in step (d) to form the formulation.

41. The method of claim 40, wherein a portion of the second preblend is co-milled prior to step (c).

42. The method of claim 40, wherein the first preblend is 4.0-14.0 weight percent magnesium stearate.

43. The method of claim 40, wherein the second preblend is 0.4-1.1 weight percent magnesium stearate.

44. A method for the preparation of a dry powder pharmaceutical formulation comprising (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile, wherein the method comprises:
(a) combining (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile, or a pharmaceutically acceptable salt thereof with magnesium stearate to form a first preblend;
(b) combining magnesium stearate and lactose to form a second preblend;
(c) blending the first and second preblends together to form the formulation; and
(d) resting the formulation for 10-120 hours.

45. The method of claim 44, wherein the resting step (d) is 15-65 hours.

46. The method of claim 44, wherein the first and second preblends are each individually co-milled prior to step (c).

47. The method of claim 44, wherein the (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate preblend is 4.0-14.0 weight percent magnesium stearate.

48. The method of claim 44, wherein the (S)-3-(3-(1-methyl-2-oxo-5-(pyrazolo[1,5-a]pyridin-3-yl)-1,2-dihydro-3H-imidazo[4,5-b]pyridin-3-yl)piperidin-1-yl)-3-oxopropanenitrile and magnesium stearate preblend is 4.0-8.0 weight percent magnesium stearate.

49. The method of claim 44, wherein the lactose and magnesium stearate preblend is 0.4-1.1 weight percent magnesium stearate.

50. The method of claim 44, wherein the lactose and magnesium stearate preblend is 0.8-1.1 weight percent magnesium stearate.

51. (canceled)

52. The method of claim 44, wherein the method comprises the additional step of:
(e) filling a capsule with the formulation.

53-66. (canceled)

67. The dry powder pharmaceutical formulation formed by the method of claim 40.
